(19) United States
(12) Patent Application Publication
(10) Pub. No.: US 2005/0107622 A1
(43) Pub. Date: May 19, 2005

(54) Novel method for wholly synthesizing of ciguatoxin ctx3c derivative

(76) Inventors: Masahiro Hirama, Sendai-shi (JP); Masayuki Inoue, Sendai-shi (JP)

Correspondence Address: Sherman & Sherman, 413 N Washington Street, Alexandria, VA, 22314, US

(21) Appl. No.: 10/498,366

(22) PCT Filed: Feb. 5, 2003
(86) PCT No.: PCT/JP03/01188

(30) Foreign Application Priority Data
Feb. 13, 2002 (JP) 2002-35075
Jun. 17, 2002 (JP) 2002-175264

(51) Int. Cl. C07D 493/14

(52) U.S. Cl. 549/341

(57) ABSTRACT

A novel method for synthesis of a novel NAP-protected ciguatoxin class precursor represented by following compound 8, and an intermediate useful in the novel method synthesizing the precursor, and a novel method of total synthesis of CTX3C class via the compound 8.

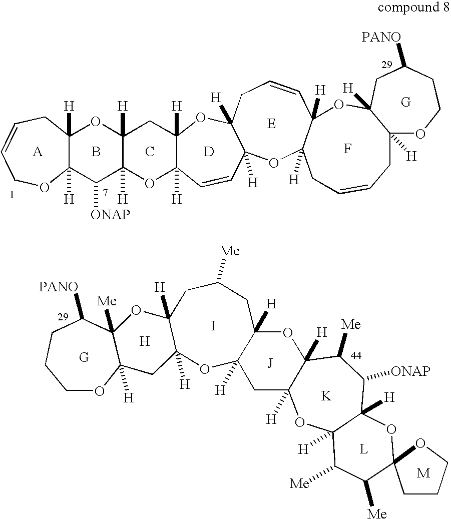

### FIELD OF THE INVENTION

The present invention relates to intermediate compounds for the synthesis of ciguatoxins and the method for synthesis thereof which can be an effective and novel route for total synthesis of ciguatoxins. In particular, the present invention relates to an intermediate which is useful for said route for total synthesis characterizing hydroxyl groups located at C7, C29 and C44 of ciguatoxin CTX3C are protected by naphthylmethyl (NAP) group (herein after shortened to NAP protected intermediate), an intermediate useful for the synthesis of NAP protected intermediate and the method for synthesis for these intermediates.

### DESCRIPTION OF THE PRIOR ART

Food-poisoning, ciguatera caused by poisoning of non-toxic fishes, widely occurs in coral reef islands region of subtropical and tropical regions, and more than 20,000 people suffer annually from ciguatera. Although the mortality is not so high, symptoms such as abnormal sensation, diarrhea, lassitude, arthralgia or itching last for several months under some circumstances. Ciguatoxins (CTX), are macromolecules characterized by fused 13 ether rings and their molecular length is approximately 3 nm. Ciguatoxins are produced from dinoflagellate Gambierdiscus toxicus and accumulate in approximately 400 kinds of fishes by means of food chain. Since toxic fishes are normal from the view points of appearance, taste and odor, it is not safe to exploit fish sources of southern sea region. Therefore, the development of detective method of ciguatoxins by means of easy and high sensitive immunological measuring method of ciguatoxins is strongly expected.

Ciguatoxins bind specifically- to voltage-dependent Na⁺ channel of neurogenic excitation membrane, activate it and generate toxicity, however, the activation mechanism of ciguatoxins at structural level is not made clear yet. Ciguatoxins exist in nature is very small and cultural production by the dinoflagellate is very slow, detail biological research and the preparation of anti-CTX antibody using natural product is virtually impossible. Under said circumstances, the quantitative supply of natural ciguatoxins by practical chemical synthesis is strongly desired.

Up to the present time, the inventors of the present invention have already proposed the total synthesis of ciguatoxin [Masahiro Hirama et al. Science. Vol. 294, pages 1904-1907 (document 1)]. However, in said total synthesis of ciguatoxin CTX3C, tribenzyl-CTX3C was synthesized by coupling ABCDE ring segments and HIJKLM ring segments and subsequent forming reaction of FG ring. However, the reaction for removing process of three benzyl protecting groups of said precursor, maintaining said completed A-M ring, is very difficult and the reaction condition is very severe. Therefore, the problem of low yield is pointed out. However, above mentioned total synthesis is the convergent total synthesis of CTX3C (document 1, FIG. 3) which is a typical ciguatoxin broadly contained in fishes, and the final deprotection is only one problem.

Therefore, the first subject of the present invention is to establish the chemical synthesis of CTX3C by new route, which solves the problem of final deprotection. Generally, in the total synthesis of natural product, when the synthesis process approaches to the final step, the numbers of surrounding atoms which affect the targeted reaction site become large and the numbers of functional groups and the molecular weight increase. Thus, the precise attention is required for the conditioning of the desired chemical conversion. Especially, the protecting group utilized for the purpose to protect a certain functional group from various reactions requires two contradicted properties, that is, chemical stability till the deprotection and the easy removal under the appropriate conditions. Therefore, the deprotection can be the most difficult process in the total synthesis. Benzyl protecting group (Bn) used in the synthesis of tribenzyl CTX3C indicated by following chemical structure, which is the precursor of CTX3C developed by the inventors of the present invention, is used under the prediction that it will be an effective protecting group for the total synthesis of CTX3C, however, there is serious difficulty at the final process.

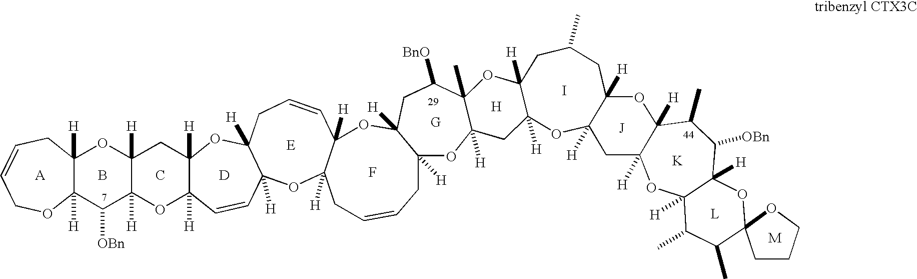

From various experimental results referring said debenzylation (Bn group), the inventors of the present invention concerned that the difficulty of debenzylation is caused by slow-reaction rate of deprotection of Bn group, and when the reaction time is prolonged, the side reaction which reduces or oxidizes allylic ether of A ring segment occurred. Therefore, the inventors of the present invention have intended to try to improve the yield of final deprotection by changing a protecting group for the total synthesis of CTX3C. In this trial, for the purpose to trace the scheme (FIG. 3 of above mentioned document 1) till final deprotection, the inventors of the present invention concerned to select the group which possesses similar chemical property to Bn group and easily deprotected than Bn group as a new protecting group.

The inventors of the present invention pay attention to p-methoxybenzyl group (MPM) and 2-naphtylmethyl group (NAP). These two groups are concerned that the side reaction (e.g. oxidization of allylic ether of above mentioned A ring segment) can be suppressed, because the oxidative deprotection of these two groups are easy when compared with that of Bn group. However, a necessary factor to be adopted as a protection group is chemical stability to each reaction shown in FIG. 3 of above mentioned document 1. The inventors of the present invention decided to judge whether each protection groups are stable or not to acetal cleavage reaction from compound 20 to compound 21, which is the most strong acid condition among the reactions shown in FIG. 3 of above mentioned document 1, by using following model compounds (M1, M2).

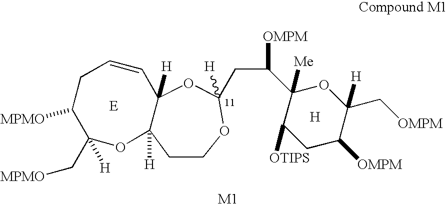

When compound M1 is provided to the acetal cleavage, removal of MPM group is progressed and desired O,S-acetal can not be obtained and only dithioacetal (compound M1′) is obtained.

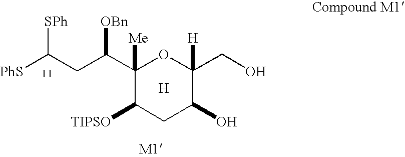

From the result, it is conjectured that MPM group is not suited as the protection group of total synthesis route of CTX3C.

In the meanwhile, when following compound M2, which is protected with NAP, is treated by Lewis acid condition, the desired compound (M2′) can be obtained by higher yield, and NAP deprotection is not progressed. Further, it becomes clear that NAP ether (compound M3) of AB ring can be deprotected (M3′) quickly at room temperature when DDQ is used, and the improvement of deprotection reaction rate at the total synthesis of CTX3C (compound prepared by deprotecting all Bn of above mentioned precursor) is expected. Thus, the inventors of the present invention decide to synthesize an intermediate for ciguatoxin CTX3C which uses NAP as a protecting group.

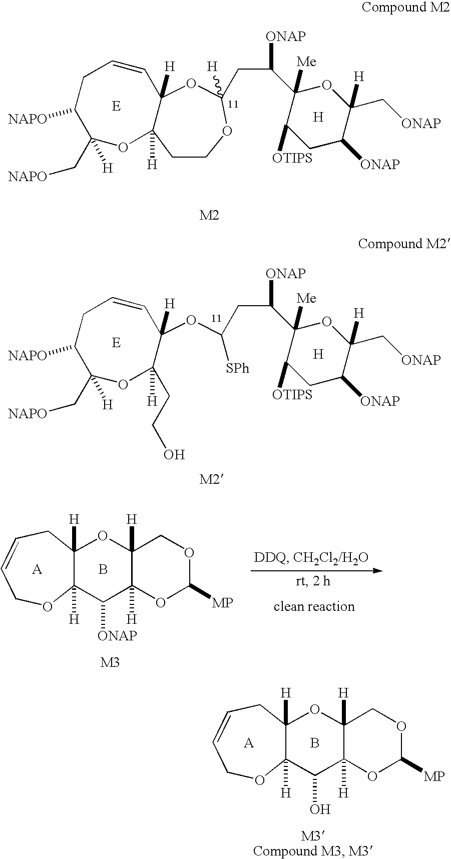

Based on the consideration about the characteristics of protecting group in above mentioned model compounds, A-E ring segment and H-M ring segment of ciguatoxins are synthesized by following scheme 1. 24-31 in scheme 1 show the generated compounds in the course of synthesizing A-E ring segment 32, and 33-35 show the generated compounds in the course of synthesizing H-M ring segment 36.

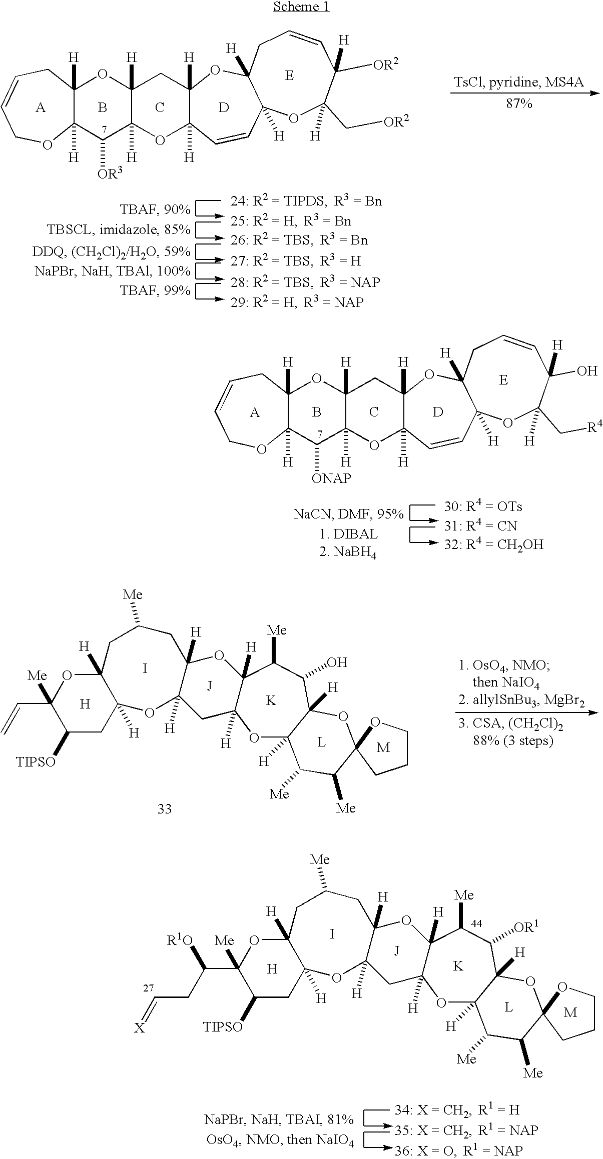

As shown in scheme 1, A-E ring segment 32 and H-M ring segment 36 characterizing each alcohol are protected by NAP group are synthesized. As already reported (above mentioned Document 1), protection groups of compound 24 are converted and the diol group 29 whose C7-OH is protected by NAP group is synthesized via 5 steps. Primary hydroxyl group of the compound 29 is selectively tosylated and a nitrile group is introduced so as to obtain the compound 31. By stepwise reduction of the compound 31, A-E ring segment 32 is obtained.

In the meanwhile, H-M ring segment is synthesized according to the aforementioned scheme 1. To aldehyde obtained by oxidation cleavage of end olefin of the compound 33, allyl tin (AllySnBu₃ in scheme 1) is worked and allylated. Since isomerization of M ring spiro ketal by MgBr₂ is observed at this process, by treating with camphor sulfonic acid (CSA in scheme 1), said compound 34 is obtained as a single diastereomer. Secondary hydroxyl group of compound 34 is protected by NAP group. After conversion of olefin to aldehyde H-M ring segment 36 is synthesized.

When the compounds 32 and 36, which are obtained in said scheme 1, are coupled according to the method disclosed in aforementioned document 1 under the presence of Sc(OTf)₃ at room temperature, acetal of following compound 37, which corresponds to the acetal (compound 20 of document 1) whose Bn group is replaced by NAP group, can be obtained.

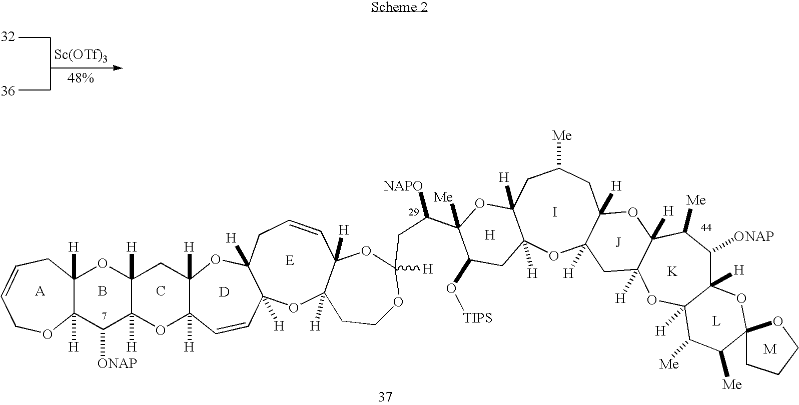

The second subject of the present invention is to provide a compound prepared by replacing the protecting group of hydroxyl groups locating at C7, C29 and C44 with naphthylmethyl (NAP) groups, which is a precursor of CTX3C, from said synthesized compound 37, to provide a method for synthesis of the precursor for synthesis of NAP protected CTX3C and to provide a method for synthesis of CTX3C from said precursor for synthesis of NAP protected CTX3C.

The inventors of the present invention considered whether it is possible to apply the synthesis route of the Bn-protected precursor compound for CTX3C described in the Document 1 to the compound 37 whose protecting group of hydroxyl group is NAP as the starting material. Consequently, for the purpose to synthesize a precursor for CTX3C having NAP as a protecting group using compound 37 as a starting material, the inventors of the present invention have found that the whole processes to synthesize a benzyl protected precursor of CTX3C described in the document 1 can be almost fully followed, and for the removal of NAP deprotecting group, specific oxidation reaction using DDQ, which is easier than the deprotecting group reaction of Bn, can be used, further, since said oxidation reaction has higher chemical structure specificity, CTX3C can be obtained by high yield. Thus the 2^{nd} subject of the present invention can be dissolved.

### DISCLOSURE OF THE INVENTION

The first one of the present invention is aiming to remove the inconvenience caused by use of Bn as the protecting group of OH groups locating at C7, C29 and C44 disclosed in the prior of Document 1, and is novel intermediate compounds, which are useful for the total synthesis of ciguatoxins, represented by compound 37, replacing synthetic route of ciguatoxins using NAP as a protecting group instead of Bn.

The second one of the present invention is the method for synthesis of the compound indicated by the compound 37 by coupling between compound 32 of the A-E ring segment and compound 36 of the H-M ring segment.

The third one of the present invention is the compound which is useful as an intermediate for the compound 37 comprising, compound 32 which has A-E ring.

The fourth one of the present invention is the compound which is useful as an intermediate for the compound 37 comprising, compound 36 which has H-M ring.

The fifth one of the present invention is a method for synthesis of the aimed compound CTX3C by total synthesis using acetal compound having naphthylmethyl (NAP) group as a protecting group for hydroxyl groups locating at C7, C29 and C44 represented by compound 37 comprising, process 1; which obtains following O,S-acetal compound 2, by placing acetal material compound 37 under the presence of 2,6-di-t-butyl-4-methylpridine, treating by trimethylsilyl trifluoromethanesulfonate (TMSOTf) and phenylthiotrimethylsilane (TMSSPh) and maintaining protecting group of NAP group,
process 2; reaction represented by following scheme 3 consisting of the process to obtain following compound 3, protecting primary hydroxyl group locating at C23 of following compound 2 with ethoxyethyl group, introducing α,β-unsaturated ester to secondary hydroxyl group locating C31, 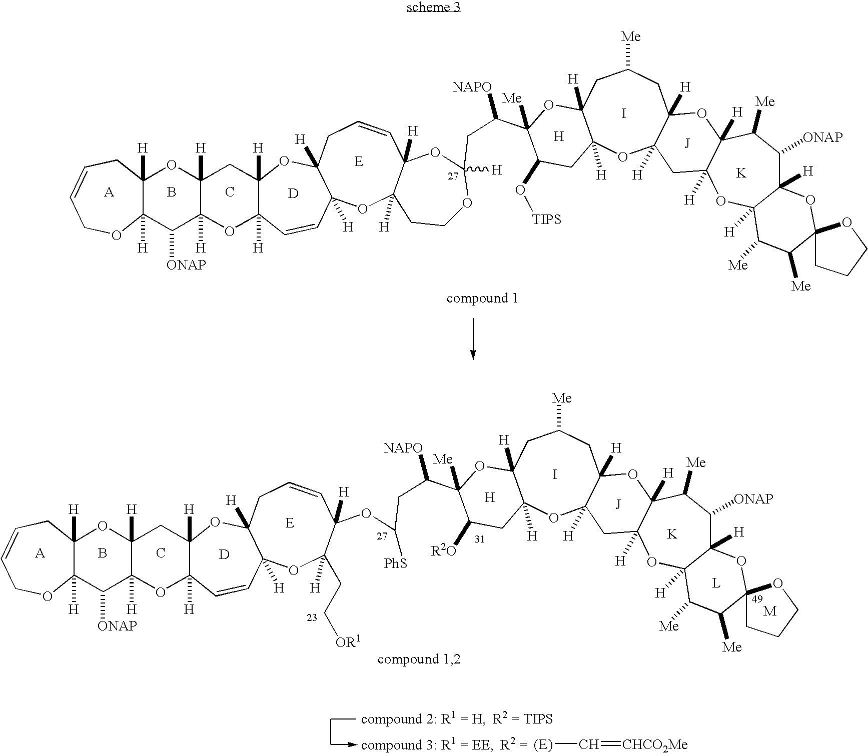
process 3; process to synthesize following compound 4 forming G ring segment by stereo selective radical cyclization of the compound 3,
process 4; process to synthesize following olefin compound 5 by reducing ester part of compound 4 and by Wittig reaction
process 5; process to obtain following compound 6 by removing ethoxyethyl group from the compound by treating under acid condition,
process 6; process to synthesize following compound 7, which is ring closing olefin metathesis substrate, by oxidizing the compound 6 and providing to Wittig reaction.
process 7; process to synthesize compound 8, which is tris NAP-CTX3C, by forming F ring using Grubbs catalyst [benzylidenebis(tricyclohexylphosphine) dichloro ruthenium] indicated by (PCy₃)₂Cl₂Ru═GhPh represented by following structural formula, 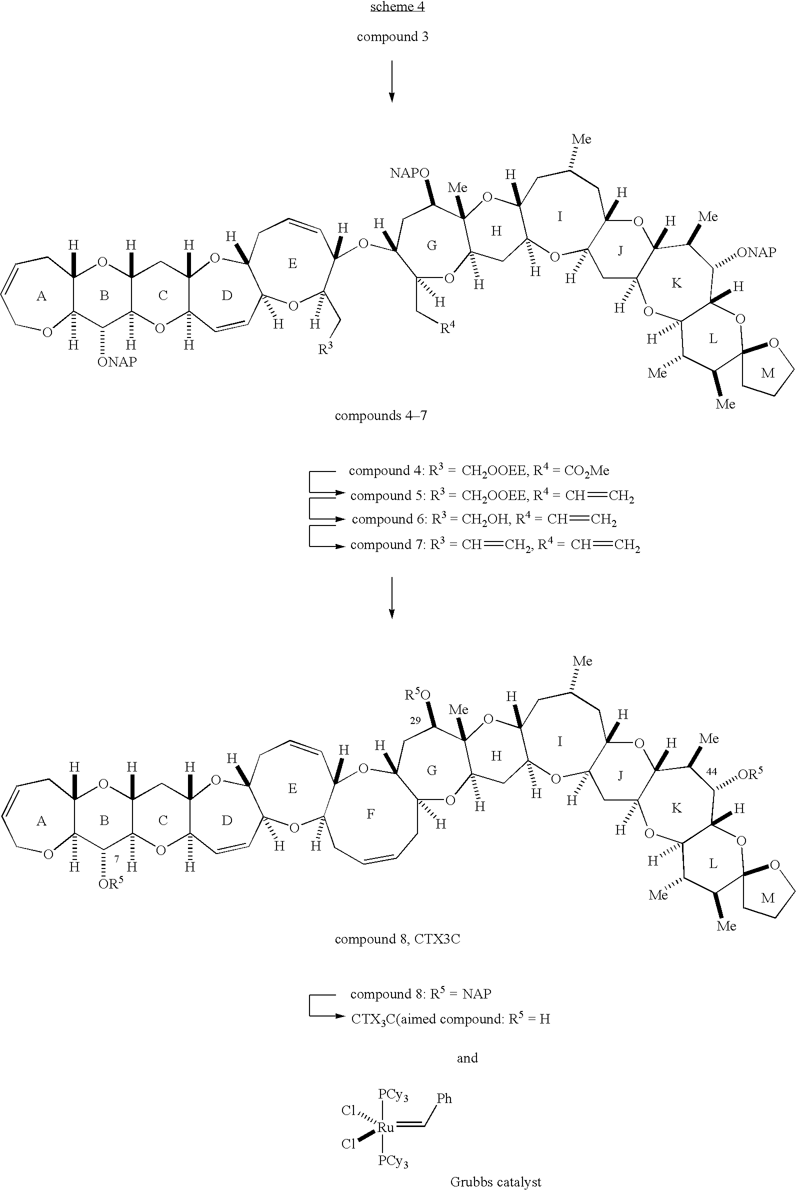
process 8; scheme 3 consisting of process to progress NAP deprotecting group reaction of compound 8 by DDQ (2,3-dichloro-5,6-dicyano-1,4-benzoquinone) under oxidation condition.

The sixth one of the present invention is a novel compound represented by following chemical formula A, in particular, a novel compound represented by following chemical formula A which is useful to synthesize CTX3C based on new route. 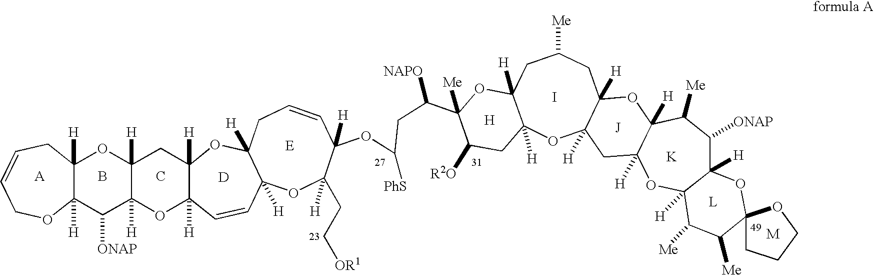
wherein, R¹ is H or EE, R² is TIPS or CH═CHCOO₂Me.

The seventh one of the present invention is a novel compound represented by the formula B, in particular, a novel compound represented by the formula B which is useful for the synthesis of CTX3C based on a new route. 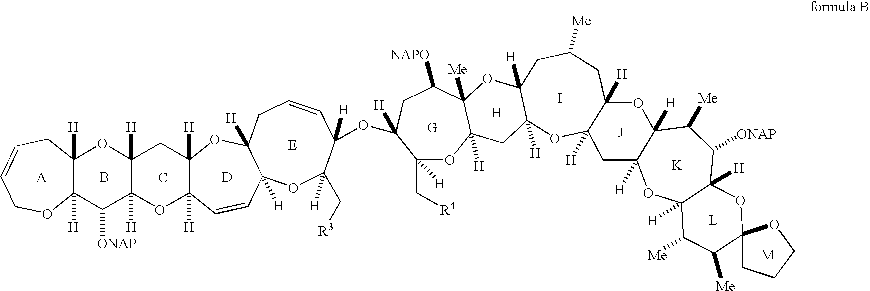
wherein, R³ is CH₂OEE, CH₂OH or CH═CH₂, R4 is COOMe or CH═CH₂.

The eighth one of the present invention is a novel compound represented by the formula C, in particular, a novel compound represented by the formula C which is useful for the synthesis of CTX3C based on a new route. 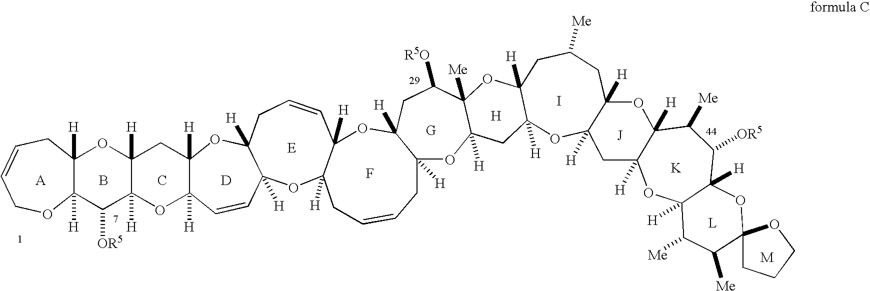
wherein, R⁵ is NAP or H.

### DESCRIPTION OF THE PREFERRED EMBODYMENT

The present invention will be illustrated more in detail.
1. The important point of the present invention is to provide intermediates for CTX3C synthesis which can dissolve the problem of deprotecting of the Bn protecting group maintaining the reaction condition of total synthesis of conventional ciguatoxins through model experiment, and to provide a method for synthesis of said intermediates.
2. For the easy understanding of the present invention, the process for the synthesis of the compound 37 is shown by scheme 5. In scheme 5, compound A, compound B and compound 1 are respectively corresponding to the compound 32 of A-E ring segment, the compound 36 of H-M ring segment and compound 37 synthesized by coupling said two compounds.

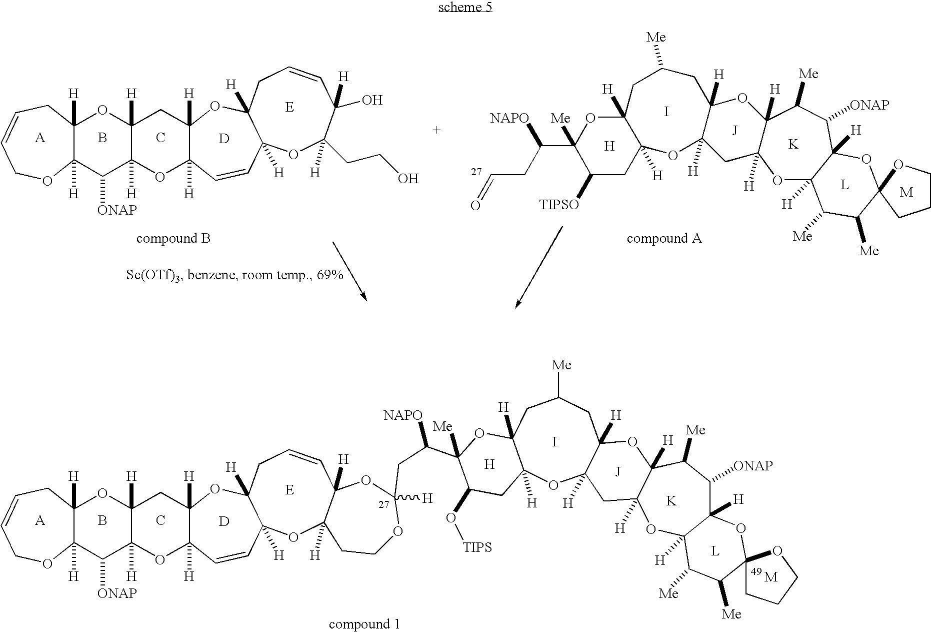

### EXAMPLES

The present invention will be illustrated more specifically according to the Examples, however, not intending to limit the scope of the claims of the present invention by the Examples.

Intermediates replaced with NAP protecting group and the process for synthesis of said intermediates are roughly described in above mentioned each schemes and in the explanation of each schemes.

### Example 1

The process for synthesis from the public known compound 24 to the final compound 32 of A-E ring segment and the physical property of the obtained intermediate are shown. The process for synthesis is shown by scheme 3 (the first half of scheme 1).

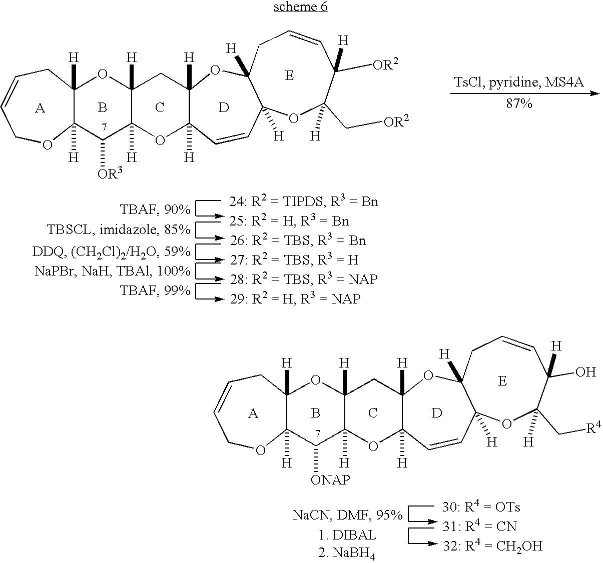

Synthesis of compound 29 prepared by converting protecting group TIPDS of compound 24 to TBS, and protecting OH group located at C7 by NAP group.

Synthesis of Diol 25:

To the THF (4 mL) solution of TBS ether 24 (97 mg, 129 μmol) TBAF (283 μL, 1.0M THF solution, 283 μmol) was added. After stirred at room temperature for 30 minutes, concentrated and purified by a silica gel column and compound 25 (59 mg, 116 μmol, 90%) was obtained.

Physical Property of the Compound 25;

¹H NMR (500 MHz, CDCl₃) δ1.51 (1H, q, J=11.3 Hz), 2.28 (1H, dt, J=11.3, 4.6 Hz), 2.26-2.31 (1H, m), 2.34 (1H, ddd, J=12.0, 5.8, 2.7 Hz), 2.34-2.48 (2H, br), 2.64 (1H, ddd, J=15.8, 7.7, 3.6 Hz), 2.69 (1H, ddd, J=12.5, 9.4, 3.8 Hz), 3.07 (1H, t, J=9.3 Hz), 3.11 (1H, td, J=9.8, 4.2 Hz), 3.24 (1H, ddd, J=11.0, 9.2, 4.8 Hz), 3.28 (1H, td, J=9.7, 4.0 Hz), 3.33 (1H, t, J=7.6 Hz), 3.38-3.43 (1H, m), 3.47 (1H, t, J=8.7 Hz), 3.60 (1H, dt, J=8.7, 3.5 Hz), 3.76-3.81 (2H, m), 3.89 (1H, dd, J=11.5, 3.9 Hz), 4.00 (1H, ddd, J=15.5, 6.0, 3.3 Hz), 4.15 (1H, ddd, J=8.8, 4.3, 2.8 Hz), 4.29 (1H, dd, J=15.5, 6.0 Hz), 4.45 (1H, dd, J=8.8, 4.0 Hz), 4.83 (1H, d, J=11.6 Hz), 4.87 (1H, d, J=11.6 Hz), 5.60 (1H, dt, J=12.4, 2.3 Hz), 5.71-5.81 (4H, m), 5.86(1H, ddd, J=11.4, 5.7, 2.7 Hz), 7.24-7.28 (1H, m), 7.30-7.35 (2H, m), 7.38-7.41 (2H, m); ¹³C NMR (125 MHz, CDCl₃) δ32.49, 34.58, 36.81, 64.45, 68.33, 70.09, 70.11, 73.15, 75.14, 78.03, 80.43, 80.78, 81.09, 82.08, 84.69, 84.96, 87.37, 126.71, 126.75, 127.42, 127.72, 128.16, 130.86, 131.33, 134.87, 136.25, 139.05.

Synthesis of bis TBS ether 26: To a DMF solution (6.0 mL), TBSCI (261 mg, 1.7 mmol) and imidazole (314 mg, 4.6 mmol) were added and stirred at 30° C. for 15 hours. After cooled down to 0° C., MeOH (100 μL) was added. Stirred for 10 minutes then diluted by ethyl acetate, and the reaction was stopped by adding saturated NaHCO₃ aqueous solution. The organic layer was washed by saturated NaHCO₃ aqueous solution and brine. After dried by MgSO₄, concentrated and purified by silica gel column, the compound 26 (73 mg, 99 μmol, 85%) was obtained.

Physical Property of the Compound 26;

[α]₂₃^{D} −42.3° (c 1.03, CHCl₃); IR (film)ν(cm⁻¹) 778, 837, 1088, 1256, 1472, 2858, 2930. ¹H NMR (500 MHz, CDCl₃) δ 0.02 (6H, s), 0.07 (6H, s), 0.88 (9H, s), 0.91 (9H, s), 1.51 (1H, q, J=11.4 Hz), 2.21-2.25 (1H, m), 2.28 (1H, dt, J=11.4, 4.2 Hz), 2.30-2.36 (1H, m), 2.63 (1H, ddd, J=11.0, 7.7, 3.7 Hz), 2.68-2.74 (1H, m), 3.06 (1H, t, J=9.3 Hz), 3.10 (1H, td, J=8.7, 3.9 Hz), 3.20-3.26 (2H, m), 3.28 (1H, dd, J=8.3, 3.6 Hz), 3.32 (1H, t, J=8.2 Hz), 3.45 (1H, t, J=8.0 Hz), 3.56 (1H, dd, J=10.8, 7.3 Hz), 3.60 (1H, dt, J=8.2, 3.2 Hz), 3.78 (1H, dd, J=8.3, 1.9 Hz), 3.94 (1H, dd, J=10.8, 2.0 Hz), 4.00 (1H, ddd, J=15.7, 5.6, 2.8 Hz), 4.07 (1H, ddd, J=8.6, 4.0, 2.3 Hz), 4.23 (1H, dd, J=9.1, 3.0 Hz), 4.28 (1H, dd, J=15.7, 5.8 Hz), 4.81 (1H, d, J=11.9 Hz), 4.87 (1H, d, J=11.9 Hz), 5.55 (1H, dt, J=12.4, 2.3 Hz), 5.64-5.68 (2H, m), 5.76 (1H, ddt, J=11.3, 8.2, 2.9 Hz), 5.86 (1H, ddd, J=11.3, 5.7, 2.6 Hz), 5.90 (1H, dt, J=12.4, 2.3 Hz), 7.24-7.28 (1H, m), 7.31-7.35 (2H, m), 7.39-7.42 (2H, m); ¹³C NMR (125 MHz, CDCl₃) δ−5.25, −5.16, −5.10, −4.40, 17.93, 18.38, 25.69, 25.96, 32.56, 34.58, 36.82, 64.28, 68.32, 70.24, 73.21, 75.12, 76.72, 77.19, 78.02, 80.59, 80.84, 80.95, 82.10, 84.79, 87.15, 87.31, 125.36, 126.76, 127.36, 127.72, 128.15, 129.99, 131.36, 135.41, 138.20, 139.16; MALDI-TOF MS Calcd for C₄₁H₆₄O₈Si₂Na (M+Na⁺) 763.4038, found 763.3288.

Synthesis of alcohol 27: Bis TBS ether (73 mg, 99 μL) was dissolved in 1,2-dichloroethane (10 mL)-H₂O (500 μL) and DDQ (24 mg, 107 μmol) was added. After stirred at 42° C. for 3.5 hours, cooled down to 0° C., and saturated Na₂S₂O₃ aqueous solution was added. Diluted by ethyl acetate, and stirred at room temperature for 1 hour. The organic layer was washed by saturated NaHCO₃ aqueous solution and brine. After dried by MgSO₄, concentrated and purified by silica gel column, the compound 26 (39 mg, 53 μmol, 55%) and compound 27 (20 mg, 31 μmol) were obtained.

Recovered compound 26 (39 mg, 53 μmol) was dissolved in 1,2-dichloroethane (5 mL)-H₂O (250 μL), and DDQ (13 mg, 31 μmol) was added. Stirred at 42° C. for 3 hours, then cooled down to 0° C., and saturated Na₂S₂O₃ aqueous solution was added. Then diluted by ethyl acetate and stirred at room temperature for 1 hour. Organic layer was washed by saturated NaHCO₃ aqueous solution and brine. After dried by MgSO₄, concentrated and purified by silica gel column, the compound 26 (7 mg, 10 μmol, 41%) and compound 27 (6 mg, 9 μmol, 39%) were obtained. Recovered compound 26 (7 mg, 10 μmol) was dissolved in 1,2-dichloroethane (2 mL)-H₂O (100 μL) and DDQ (2.4 mg, 11 μmol) was added. Stirred at 42° C. for 3 hours, then cooled down to 0° C., and saturated Na₂S203 aqueous solution was added. Then diluted by ethyl acetate and stirred at room temperature for 1 hour. Organic layer was washed by saturated NaHCO₃ aqueous solution and brine. After dried by magnesium sulfate anhydride, concentrated and purified by silica gel column, the compound 27 (4 mg, 6 μmol, 59%) was obtained. Debenzylation reaction was repeated for 4 times and total 47 mg (72 μmol) of the compound 27 was obtained.

Physical Property of the Compound 27;

[αα]₂₃^{D} −59.1° (c 0.93, CHCl₃); IR (film)ν(cm⁻¹) 778, 836, 1090, 1256, 1472, 2360, 2858, 2930, 3475; ¹H NMR (500 MHz, CDCl₃) δ 0.02 (6H, s), 0.07 (6H, s), 0.88 (9H, s), 0.91 (9H, s), 1.53 (1H, q, J=11.3 Hz), 2.22 (1H, ddd. J=17.7, 5.3, 2.5 Hz), 2.28 (1H, dt, J=11.3, 4.3 Hz), 2.34-2.41 (1H, m), 2.62 (1H, ddd, J=16.4, 8.3, 2.9 Hz), 2.70 (1H, ddd, J=17.7, 8.3, 4.0 Hz), 2.71-2.28 (1H, br), 3.00 (1H, t, J=9.3 Hz), 3.11 (1H, ddd, J=12.5, 8.6, 4.2 Hz), 3.20-3.27 (3H, m), 3.31 (1H, ddd, J=8.9, 7.6, 2.0 Hz), 3.56 (1H, dd, J=10.7, 7.6 Hz), 3.57-3.64 (2H, m), 3.80 (1H, ddd, J=8.9, 3.4, 2.1 Hz), 3.91 (1H, dd, J=10.7, 1.4 Hz), 4.01 (1H, ddd, J=15.5, 6.3, 3.8 Hz), 4.06 (1H, ddd, J=10.0, 4.7, 3.0 Hz), 4.22 (1H, dd, J=8.9, 2.7 Hz), 4.32 (1H, dd, J=15.5, 6.0 Hz), 5.60 (1H, dt, J=12.4, 2.3 Hz), 5.64-5.67 (2H, m), 5.84 (1H, ddt, J=11.6, 8.8, 2.9 Hz), 5.88-5.94 (2H, m); ¹³C NMR (125 MHz, CDCl₃) δ −5.24, −5.18, −5.13, −4.41, 17.94, 18.35, 25.72, 25.92, 0.32.56, 34.28, 36.73, 64.25, 68.15, 70.19, 73.02, 74.23, 75.88, 77.92, 80.02, 80.61, 80.99, 84.78, 87.23, 87.61, 125.31, 127.69, 129.78, 131.65, 135.58, 138.29; MALDI-TOF MS Calcd for C₃₄H₅₈O₈Si₂Na (M+Na⁺) 673.3568, found 673.3409.

NAP ether 28: THF (1 mL)-DMF (300 μL) solution of alcohol 27 (47 mg, 72 μmol) was cooled down to 0° C., then TBAI (1.3 mg, 3.6 μmol), NaH (3.5 mg, 144 μmol) and NAPBr (24 mg, 108 μmol) were added, and stirred at 30° C. for 4 hours. Cooled down to 0° C., MeOH (100 μL) was added and stopped the reaction by adding saturated NH₄Cl aqueous solution and brine. After dried by MgSO₄, concentrated and purified by silica gel column, the compound 28 (58 mg, 72 μmol, quant) was obtained.

Physical Property of the Compound 28;

[α]₂^{D} −33.6° (c 1.00, CHCl₃); IR (film)ν(cm⁻¹) 777, 836, 1087, 1256, 2341, 2360, 2857, 2929; ¹H NMR (500 MHz, CDCl₃) δ0.02 (6H, s), 0.07 (6H, s), 0.88 (9H, s), 0.91 (9H, s), 1.51-1.57 (1H, m), 2.20-2.25 (1H, m), 2.28 (1H, dt, J=11.8, 3.9 Hz), 2.31-2.38 (1H, m), 2.64 (1H, ddd, J=16.0, 7.6, 3.8 Hz), 2.71 (1H, ddd, J=16.6, 8.8, 3.8 Hz), 3.09-3.12 (2H, m), 3.24 (1H, ddd, J=11.0, 8.7, 4.9 Hz), 3.28 (1H, td, J=9.5, 3.8 Hz), 3.34 (1H, ddd, J=9.3, 7.3, 2.2 Hz), 3.36 (1H, t, J=8.7 Hz), 3.51 (1H, t, J=8.8 Hz), 3.56 (1H, dd, J=10.7, 7.3 Hz), 3.61 (1H, dt, J=8.8, 2.8 Hz), 3.81 (1H, ddd, J=8.7, 4.5, 2.8 Hz), 3.94 (1H, dd, J=10.7, 2.2 Hz), 4.04 (1H, ddd, J=15.3, 5.3, 3.0 Hz), 4.08 (1H,ddd, J=8.8, 4.4, 2.6 Hz), 4.23 (1H, dd, J=9.3, 2.4 Hz), 4.31 (1H, dd, J=15.3, 5.8 Hz), 4.97 (1H, d, J=11.9 Hz), 5.04 (1H, d, J=11.9 Hz), 5.60 (1H, dt, J=12.0, 2.3 Hz), 5.65-5.68 (2H, m), 5.77 (1H, ddt, J=11.9, 7.6, 4.1 Hz), 5.86 (1H, ddd, J=11.9, 5.7, 2.8 Hz), 5.91 (1H, dt, J=12.0, 2.2 Hz), 7.44-7.46 (2H, m), 7.53-7.55 (1H, m), 7.80-7.83 (3H, m), 7.84-7.86 (1H, m); ¹³C NMR (125 MHz, CDCl₃) δ −5.19, −5.12, −5.09, −4.39, 17.95, 18.42, 25.73, 25.99, 32.60, 34.63, 36.87, 64.32, 67.96, 68.36, 70.30, 73.26, 75.14, 78.04, 80.67, 80.88, 81.01, 82.09, 84.82, 87.22, 87.40, 125.38, 125.58, 125.83, 126.06, 126.29, 126.77, 127.63, 127.80, 127.87, 130.03, 131.35, 132.95, 133.28, 135.47, 136.71, 138.21; MALDI-TOF MS Calcd for C₄₅HG₆O₈Si₂Na (M+Na⁺) 813.4194, found 813.3141.

Synthesis of diol 29: To THF solution (2 mL) of NAP ether 28 (58 mg, 72 μmol), TBAF (158 mL, 1.0M THF solution, 158 μmol) was added. After stirred at room temperature for 2 hours, concentrated, purified by silica gel column and compound 29 (41 mg, 72 μmol, 99%) was obtained.

Physical Property of the Compound 29

[α]₂^{D} −61.1° (c 0.82, CHCl₃); IR (film)ν(cm⁻¹) 756, 1093, 1260, 1367, 2360, 2875, 3399; ¹H NMR (500 MHz, CDCl₃) δ1.52 (1H, q, J=11.3 Hz), 2.18-2.24 (2H, br), 2.25-2.38 (3H, m), 2.63 (1H, ddd, J=15.8, 7.6, 4.8 Hz), 2.67 (1H, ddd, J=13.0, 9.2, 3.4 Hz), 3.09-3.12 (2H, m), 3.24 (1H, ddd, J=11.3, 8.8, 4.4 Hz), 3.28 (1H, td, J=10.2, 4.3 Hz), 3.37 (1H, t, J=8.7 Hz), 3.41 (1H, hep, J=4.8 Hz), 3.52 (1H, t, J=8.7 Hz), 3.61 (1H, dt, J=8.8, 3.0 Hz), 3.77-3.83 (2H, m), 3.90 (1H, dd, J=10.3, 2.2 Hz), 4.04 (1H, ddd, J=15.3, 6.2, 3.0 Hz), 4.14 (1H, ddd, J=9.0, 4.0, 2.4 Hz), 4.31 (1H, dd, J=15.2, 5.7 Hz), 4.46 (1H, dd, J=8.6, 4.5 Hz), 4.98 (1H, d, J=11.8 Hz), 5.02 (1H, d, J=11.8 Hz), 5.64 (1H, dt, J=12.5, 2.2 Hz), 5.71-5.81 (4H, m), 5.86 (1H, ddd, J=11.6, 6.0, 3.6 Hz), 7.43-7.48 (2H, m), 7.52-7.56 (1H, m), 7.79-7.85 (4H, m); ¹³C NMR (125 MHz, CDCl₃) δ32.50, 34.59, 36.83, 64.48, 68.36, 70.20, 73.17, 75.15, 78.03, 80.47, 80.81, 81.09, 82.01, 84.70, 84.99, 87.46, 125.63, 125.87, 126.06, 126.30, 126.76, 126.84, 127.64, 127.84, 127.86, 130.89, 131.36, 132.96, 133.27, 134.89, 136.22, 136.62; MALDI-TOF MS Calcd for C₃₃H₃₈O₈Na (M+Na⁺) 585.2465, found 585.2108.

Synthesis of Compounds from 30 to 32

Synthesis of tosyl 30: MS 4 Å (20 mg) was added to pyridine solution (2 mL) of diol 29 (41 mg, 72 μmol) and stirred at room temperature for 1 hour. TsCl (15 mg, 80 μmol) was added and stirred at 40° C. for 3 hours. Cooled down to 0° C., and the reaction was, stopped by adding saturated NaHCO₃ aqueous solution. Organic layer was washed by saturated NH₄Cl aqueous solution and brine. After dried by MgSO₄, concentrated and purified by silica gel column, compound 29 (15 mg, 27 μmol, 37%) and compound 30 (25 mg, 34 μmol, 47%) were obtained.

Recovered compound 29 (15 mg, 27 μmol) was dissolved in pyridine (1 mL) and MS 4 Å (20 mg) was added. After stirred at room temperature for 1 hour, TsCl (4 mg, 21 μmol) was added and stirred at 40° C. for 3 hours. Cooled down to 0° C., and the reaction was stopped by dropping saturated NaHCO₃ aqueous solution. Organic layer was washed by saturated NH₄Cl aqueous solution and brine. After dried by MgSO₄, concentrated and purified by silica gel column, compound 29 (4 mg, 8 μmol, 30%) and compound 30 (11 mg, 15 μmol, 62%) were obtained.

Recovered compound 29 (4 mg, 8 μmol) was dissolved in pyridine (400 μL) and MS 4 Å (20 mg) was added. After stirred at room temperature for 1 hour, TsCl (2 mg, 10 μmol) was added and stirred at 40° C. for 3 hours. Cooled down to 0° C., and the reaction was stopped by adding saturated NaHCO₃ aqueous solution. Organic layer was washed by saturated NH₄Cl aqueous solution and brine. After dried by MgSO₄, concentrated and purified by silica gel column, compound 30 (5 mg, 7 μmol, 87%) was obtained.

Tosylation reaction was repeated 3 times as mentioned above, total 40 mg (56 μmol) of compound 30 was obtained.

Physical Property of Compound 30;

[α]₂₃D −67.4° (c 0.75, CHCl₃); IR (film)ν(cm⁻¹) 668, 756, 1094, 1176, 1363, 2341, 2360, 2878, 3027, 3438; ¹H NMR (500 MHz, CDCl₃) δ1.50 (1H, q, J=11.3 Hz), 2.19-2.37 (4H, m), 2.58-2.66 (2H, m), 3.13-3.14 (2H, m), 3.21 (1H, ddd, J=11.3, 8.8, 4.6 Hz), 3.28 (1H, td, J=9.5, 4.0 Hz), 3.46 (1H, t, J=8.7 Hz), 3.47 (1H, dt, J=9.3, 3.6 Hz), 3.51 (1H, t, J=8.7 Hz), 3.36 (1H, dt, J=8.3, 3.4 Hz), 3.78 (1H, ddd, J=9.0, 4.6, 2.5 Hz), 4.01-4.06 (2H, m), 4.28-4.34 (2H, m), 4.36-4.41 (1H, m), 4.98 (1H, d, J=12.0 Hz), 5.03 (1H, d, J=12.0), 5.60 (1H, dt, J=12.8, 0.2.7 Hz), 5.68 (1H, dd, J=11.0, 5.2 Hz), 5.74 (1H, dt, J=12.9, 2.4 Hz), 5.75-5.81 (2H, m), 5.60 (1H, ddd, J=11.4, 6.0, 3.0 Hz), 7.34-7.37 (2H, m), 7.44-7.48 (2H, m), 7.54-7.56 (1H, m), 7.80-7.84 (7H, m); ¹³C NMR (125 MHz, CDCl₃) δ21.66, 32.41, 34.58, 36.77, 68.34, 68.48, 71.19, 73.12, 75.16, 76.73, 77.95, 80.47, 80.79, 81.45, 81.95, 83.18, 84.46, 87.47, 125.64, 125.87, 126.07, 126.32, 126.78, 127.42, 127.65, 127.87, 128.00, 129.84, 129.90, 130.68, 130.92, 131.38, 131.39, 132.90, 132.96, 133.23, 134.75, 135.47, 136.59; MALDI-TOF MS Calcd for C₄₀H₄₄SO₁₀Na (M+Na⁺) 739.2552, found 739.1758.

Synthesis of nitrile 31: NaCN (27 mg, 560 μmol) was added to DMF solution (2 mL) of tosyl 30 (40 mg, 56 μmol) and stirred at 42° C. for 20 hours. Cooled down to 0° C., and the reaction was stopped by dropping saturated NaHCO₃ aqueous solution. Organic layer was washed by saturated NH₄Cl aqueous solution and brine. After dried by MgSO₄, concentrated and purified by silica gel column, compound 31 (31 mg, 53 μmol, 95%) was obtained.

Physical Property of Compound 31;

[α]₂₆^{D} −52.1° (c 0.61, CHCl₃); IR (film)ν(cm⁻¹) 687, 757, 1092, 2067, 2342, 2360, 2873, 3385; ¹H NMR (500 MHz, CDCl₃) δ1.50 (1H, q, J=11.3 Hz), 2.26-2.38 (4H, m), 2.59 (1H, dd, J=16.5, 8.0 Hz), 2.60-2.71 (3H, m), 2.91 (1H, dd, J=16.5, 2.6 Hz), 3.09-3.13 (2H, m), 3.24 (1H, ddd, J=11.0, 9.2, 5.0 Hz), 3.28 (1H, td, J=9.6, 3.9 Hz), 3.37 (1H, t, J=8.4 Hz), 3.52 (1H, t, J=8.4 Hz), 3.58 (1H, td, J=8.8, 2.9 Hz), 3.63 (1H, dt, J=8.7, 3.1 Hz), 3.81 (1H, ddd, J=8.7, 4.9, 2.4 Hz), 4.05 (1H, ddd, J=15.4, 5.0, 2.6 Hz), 4.14 (1H, ddd, J=8.0, 4.6, 2.7 Hz), 4.26 (1H, dd, J=8.3, 6.0 Hz), 4.31 (1H, dd, J=10.6, 5.9 Hz), 4.98 (1H, d, J=11.6 Hz), 5.03 (1H, d, J=11.6 Hz), 5.67 (1H, dt, J=12.6, 2.1 Hz), 5.70 (1H, dd, J=10.5, 5.2 Hz), 5.77 (1H, ddt, J=11.3, 7.9, 2.4 Hz), 5.80-5.90 (2H, m), 5.96 (1H, dt, J=12.7, 2.2 Hz), 7.44-7.49 (2H, m), 7.53-7.56 (1H, m), 7.80-7.84 (4H, m); ¹³C NMR (125 MHz, CDCl₃) 522.88, 32.48, 34.59, 36.82, 40.93, 68.35, 71.32, 73.12, 75.17, 77.98, 80.48, 80.82, 81.35, 81.37, 81.89, 84.14, 87.48, 118.22, 125.64, 125.88, 126.11, 126.36, 126.74, 127.65, 128.39, 131.01, 131.37, 132.98, 133.26, 134.27, 135.35, 136.54; MALDI-TOF MS Calcd for C₃₄H₃₇NO₇Na (M+Na⁺) 594.2467, found 594.1830.

Synthesis of diol 32: Nitrile 31 (31 mg, 53 μmol) was dissolved in CH₂Cl₂ (2 mL) and cooled down to −78° C. DIBAL (84 μL, 0.95M hexane solution, 80 μmol) was added slowly and stirred for 30 minutes during the temperature was elevated to −53° C. The reaction was stopped by adding ethyl acetate and saturated NH₄Cl aqueous solution. Organic layer was washed by saturated NaHCO₃ aqueous solution and brine. After dried by MgSO₄, concentrated and purified by silica gel column, hemiacetal (29 mg, 51 μmol, 96%) was obtained.

Hemiacetal (29 mg, 51 μmol) was dissolved in MeOH (1 mL)-CH₂Cl₂ (2 mL) and cooled down to −50° C. NaBH₄ (19 mg, 510 μmol) was added then stirred for 1 hour, while the temperature was elevated to room temperature. Cooled down to 0° C., and the reaction was stopped by adding 500 μL of 0.02N HCl. Organic layer was washed by saturated NaHCO₃ aqueous solution and brine. After dried by MgSO₄, concentrated, purified by silica gel column, compound 32 was obtained.

Physical Property of Compound 32;

[α]₂₆^{D} −36.7° (c 0.24, CHCl₃); ¹H NMR (500 MHz, CDCl₃) δ1.50 (1H, q, J=11.3 Hz), 1.87 (1H, dtd, J=14.4, 7.5, 4.6 Hz), 2.10 (1H, dtd, J=11.0, 7.5, 4.5 Hz), 2.25-2.38 (3H, m), 2.64 (1H, ddd, J=15.8, 8.2, 4.0 Hz), 2.72 (1H, ddd, J=13.3, 9.6, 3.0 Hz), 3.08-3.13 (2H, m), 3.23 (1H, ddd, J=11.3, 9.0, 4.8 Hz), 3.28 (1H, td, J=9.6, 3.7 Hz), 3.37 (1H, t, J=8.4 Hz), 3.53 (1H, t, J=8.2 Hz), 3.50-3.56 (1H, m), 3.59 (1H, dt, J=8.6, 3.0 Hz), 3.83 (1H, ddd, J=9.0, 4.7, 2.3 Hz), 3.85 (1H, ddd, J=14.0, 7.5, 3.5 Hz), 3.92 (1H, ddd, J=11.0, 7.0, 3.5 Hz), 4.04 (1H, ddd, J=15.3, 4.9, 2.6 Hz), 4.15 (1H, ddd, J=8.6, 4.3, 2.1 Hz), 4.31 (1H, dd, J=15.3, 6.2 Hz), 4.31-4.33 (1H, m), 4.98 (1H, d, J=11.3 Hz), 5.02 (1H, d, J=11.3 Hz), 5.64 (1H, dt, J=12.7, 2.4 Hz), 5.72-5.81 (4H, m), 7.44-7.48 (2H, m), 7.53-7.55 (1H, m), 7.80-7.83 (4H, m); ¹³C NMR (125 MHz, CDCl₃) δ32.52, 34.64, 35.39, 36.89, 59.75, 68.39, 71.62, 73.22, 75.12, 76.80, 78.03, 80.56, 80.89, 81.32, 82.03, 84.58, 84.74, 87.46, 125.61, 125.86, 126.08, 126.28, 126.29, 126.58, 126.76, 127.65, 127.83, 127.88, 130.99, 131.33, 132.99, 133.31, 134.81, 136.34.

### Example 2

Method for synthesis of compounds from 33 to 36, which is the final product of H-M ring segment, and the physical properties of the obtained intermediate are shown. Synthesis process is shown as scheme 4 (latter half of scheme 1).

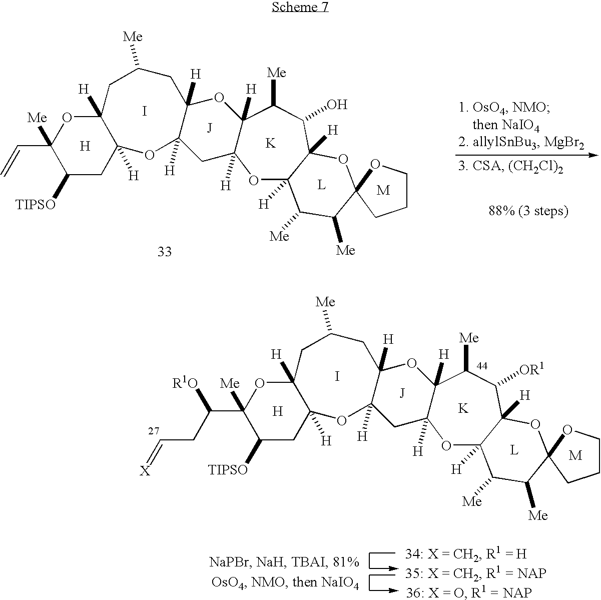

Synthesis of homoallylalchol 34: Olefin 33 (66.5 mg, 96.0 μL) and NMO (0.2 mL, 50% aqueous solution, 0.96 mmol) were dissolved in t-BuOMe (1.0 mL)-t-BuOH (3.0 mL)-H₂O (11.0 mL) and OsO4 (250 μL, 19 mM t-BuOH solution, 4.8 μmol) was added. After stirred at room temperature for 3 days, pH7 phosphoric acid buffer (1 mL) and NaIO₄ (109 mg, 0.51 mmol) were added and stirred at room temperature for another 2 hours. In order to stop the reaction, NaIO₄ (101 mg, 0.47 mmol) was further added, and the reaction was stopped by Na₂S₂O₃ aqueous solution (5 mL). Solution was extracted by ethyl acetate for 3 times, organic layer was washed by brine, then dried by MgSO₄. After concentrated, purified by Florisil column and crude aldehyde was obtained, was prepared by treating Mg turning (47 mg, 1.9 mmol) in Et₂O (11.0 mL) with 1,2-dibromoethane (0.17 mL, 1.9 mmol), stirring from 0° C. to room temperature for 1 hour and by evaporating solution. To the obtained MgBr₂, CH₂Cl₂ (1.5 mL) solution of said crude aldehyde was added at room temperature. After stirred, at room temperature for 15 minutes, the solution was cooled down to −80° C., allyltributyltin (90 μL, 0.29 mmol) was introduced into solution and the temperature was elevated to 10° C., by 9 hours. The reaction was stopped by adding saturated NH₄Cl aqueous solution and extracted by ethyl acetate for 3 times. Combined organic layer was washed by brine, and dried by MgSO₄. After concentrated, purified by a silica gel pad, and desired compound was obtained as a mixture of C29- and C49-epimer. To 1,2-dichloroethane (2.0 mL) solution of said crude product, CSA (6.9 mg, 30 μmol) was added, and stirred at room temperature for 2 hours. Reaction solution was diluted by ethyl acetate, NaHCO₃ aqueous solution was added, further extracted by ethyl acetate for 3 times. Combined organic layer was washed by brine, and dried by MgSO₄. After concentration, purified by flash column and the compound 34 was obtained as C29-epimer mixture (62.0 mg, 84.1 μmol, 29S=˜10:1, 88%, 3 steps).

Physical Property of Compound 34

[α]_{D}²² −21.3 (c 1.240, CHCl₃); IR (film)ν3488, 2945, 2868, 1640, 1462, 1380, 1099, 1075, 1023, 918, 883, 757, 683 cm⁻¹; ¹H NMR (500 MHz, CDCl₃) δ0.90 (3H, d, J=6.5 Hz, Me57), 1.04 (3H, d, J=47.5 Hz, Me54), 1.05-1.09 (6H, m, Me53, Me56), 1.06 (21H, s, TIPS), 1.16 (3H, d, J=7.5 Hz, Me55), 1.42 (1H, q, J=12.0 Hz, H40), 1.44-1.63 (4H, m, H35, 37, 47, 48), 1.70 (1H, q, J=12.0 Hz, H32), 1.72-2.04 (8H, m, H35, 36, 37, 43, 50×2, 51×2), 2.07 (1H, dt, J=12.5, 5.0 Hz, H32), 2.17-2.28 (2H, m, H28, H40), 2.32-2.39 (1H, m, H28), 2.84 (1H, dd, J=9.5, 5.5 Hz, H42), 2.96-3.02 (1H, m, H38), 3.07 (1H, td, J=10.5, 5.0 Hz, H33), 3.15 (1H, ddd, J=9.0, 6.5, 5.0 Hz, H39), 3.26 (1H, t, J=10.0 Hz, H46), 3.30 (1H, td, J=10.0, 3.0 Hz, H34), 3.60 (1H, td, J=0.5, 3.0 Hz, H29), 3.66 (1H, bd, J=9.5 Hz, H45), 3.67-3.73 (2H, m, H41, 44), 3.79 (1H, q, J=7.5 Hz, H52), 3.89 (1H, td, J=7.5, 4.5 Hz, H52), 4.17 (1H, dd, J=12.0, 5.0 Hz, H31), 5.04-5.11 (2H, m, H26×2), 5.92 (1H, ddd, J=17.0, 10.0, 7.0 Hz, H27); ¹³C NMR (125 MHz, CDCl₃) δ12.78 (×3), 13.41, 13.58, 15.82, 18.10 (×3), 18.17 (×3), 19.60, 24.24, 27.56, 28.07, 34.89, 35.72, 38.01, 38.50, 40.50, 42.11 (×2), 45.41, 45.83, 67.53, 67.63, 71.65, 72.65, 73.34, 75.50, 77.11, 78.15, 78.28, 81.10, 83.13, 83.20, 86.15, 108.63, 116.51, 136.44; MALDI-TOF MS, calcd. for C₄₁H₇₂O₉SiNa 759.4844 (M+Na⁺), found 759.4928, calcd. for C₄₁H₇₂O₉SiK 775.4583 (M+K⁺), found 775.4726.

Synthesis of bis NAP ether 35: Diol 34 (69.3 mg, 94.0 μmol) was dissolved in mixed solution of THF (1.8 mL)-DMF (0.6 mL), then NaH (36 mg, 60% oil suspension, 0.9 mmol), NAPBr (83 mg, 0.38 mmol) and TBAI (15 mg, 41 μmol) were added in order at 0° C. After stirred at room temperature for 1 day, MeOH was added to the reaction solution and extracted by ethyl acetate for 3 times. Combined organic layer was washed by brine and saturated NH₄Cl aqueous solution and dried on MgSO₄. After concentrated, purified by a flash column and compound 35 (77.2 mg, 75.9 μmol, 81%) was obtained.

Physical Property of Compound 35;

[α]_{D}²⁶ 1.9 (c 0.953, CHCl₃); IR (film)ν3056, 2944, 2866, 1509, 1460, 1377, 1330, 1098, 1074, 1027, 883, 815, 757 cm⁻¹; ¹H NMR (500 MHz, CDCl₃) δ0.90-1.08 (12H, m, Me54, 56, 57, 58), 1.02 (21H, s, TIPS), 1.12 (3H, d, J-=7.5 Hz, Me55), 1.15 (3H, s, Me53), 1.40 (1H, q, J=12.0 Hz, H40), 1.51-1.62 (4H, m, H35, 37, 47, 48), 1.73 (1H, q, J=11.5 Hz, H32), 1.75-2.02 (7H, m, H35, 36, 37, 50×2, 51×2), 2.11 (1H, dt, J=12.5, 5.0 Hz, H32), 2.17-2.24 (2H, m, H40, H43), 2.38-2.62 (2H, m, H28×2), 2.87 (1H, dd, J-9.0, 4.5 Hz, H42), 2.97-3.03 (1H, m, H38), 3.04-3.14 (2H, m, H33, 39), 3.31 (1H, td, J=10.0, 2.5 Hz, H34), 3.43 (1H, t, J=9.5 Hz, H46), 3.50 (1H, d, J=3.0 Hz, H44), 3.65-3.69 (2H, m, H29, 45), 3.76 (1H, q, J=7.5 Hz, H52), 3.83-3.91 (2H, m, H41, 52), 4.27 (1H, dd, J=12.0, 5.0 Hz, H31), 4.74 (1H, d, J=11.5 Hz, NAP), 4.80 (1H, d, J=12.0 Hz, NAP), 4.84 (1H, d, J=12.0 Hz, NAP), 4.84 (1H, d, J=12.0 Hz, NAP), 5.02 (1H, bd, J=10.0 Hz, H26), 5.13 (1H, bd, J=17.0 Hz, H26), 5.99 (1H, ddd, J=17.0, 10.0, 7.0 Hz, H27), 7.39-7.49 (5H, m, NAP), 7.52-7.56 (1H, m, NAP), 7.75-7.84 (8H, m, NAP); ¹³C NMR (125 MHz, CDCl₃) δ13.05 (×3), 13.45, 13.94, 15.97, 18.31 (×3), 18.32 (×3), 19.93, 24.34, 27.56, 28.07, 33.25, 35.03, 38.38, 38.57, 40.28, 40.57, 41.86, 45.52, 46.02, 67.35, 68.38, 71.50, 71.81, 72.16, 73.46, 74.19, 77.99, 79.99, 80.87, 81.95, 82.97, 83.07, 84.66, 86.73, 108.26, 115.87, 125.40, 125.60, 125.73, 125.75 (×2), 125.92, 126.02, 126.08, 127.56 (×2), 127.65, 127.69, 127.74, 127.79, 132.70, 132.81, 133.20, 133.25, 136.88, 136.99, 137.25; MALDI-TOF MS, calcd. for C₆₃H₈₈O₉SiNa 1039.6096 (M+Na⁺), found 1039.5963, calcd. for C₆₃H₈₈O₉SiK 1055.5835 (M+K⁺), found 1055.5853.

Synthesis of aldehyde 36: Olefin 35 (17.9 mg, 17.6 μmol) and NMO (73 μL, 50% aqueous solution, 0.35 mmol) were dissolved in mixed solvent of t-BuOMe (0.45 mL)-t-BuOH (0.45 mL)-H₂O (0.15 mL), and OSO₄ (46 μL, 19 mM t-BuOH solution, 0.88 μmol) were added at room temperature. After stirred at room temperature, pH7 phosphoric acid buffer (1 mL) and NaIO₄ (36 mg, 0.17 mmol) were added and stirred at room temperature for 1 day. The reaction was stopped by saturated Na₂S₂O₂ aqueous solution. Solution was extracted by ether for 3 times, combined organic layer was washed by brine and dried by MgSO₄. After concentrated, purified by Florisil Pad, crude aldehyde 36 was obtained.

Physical Property of Compound 36;

¹H NMR (500 MHz, CDCl₃) δ0.88-0.93 (3H, m), 1.01-1.08 (9H, m), 1.05 (21H, s), 1.10-1.15 (3H, m), 1.39 (0.5H, q, J=11.5 Hz), 1.40 (0.5H, q, J=11.5 Hz), 1.47-1.57 (5H, m), 1.68-2.02 (10H, m), 2.04-2.36 (8H, m), 2.52-2.59 (0.5H, m), 2.59-2.67 (0.5H, m), 2.64 (1H, ddd, J=16.0, 8.0, 4.0 Hz), 2.86 (1H, dd, J=8.5, 4.0 Hz), 2.96-3.02 (1H, m), 3.05-3.16 (4H, m), 3.19-3.31 (3H, m), 3.37 (0.5H, t, J=9.0 Hz), 3.37 (0.5H, t, J=9.0 Hz), 3.42 (0.5H, t, J=9.5 Hz), 3.43 (0.5H, t, J=9.5 Hz), 3.46-3.60 (4H, m), 3.67 (1H, d, J=10.0 Hz), 3.64-3.90 (7H, m), 4.00-4.14 (3H, m), 4.21 (0.5H, dd, J=11.0, 5.0 Hz), 4.30 (1H, dd, J=15.5, 5.5 Hz), 4.31-4.46 (0.5H, m), 4.67-4.70 (0.5H, m), 4.70 (0.5H, d, J=11.5 Hz), 4.71 (0.5H, d, J=11.5 Hz), 4.79 (1H, d, J=12.0 Hz), 4.81-4.87 (2H, m), 4.90 (0.5H, d, J=11.5 Hz), 4.98 (0.5H, d, J=12.0 Hz), 4.99 (0.5H, d, J=12.0 Hz), 5.03 (0.5H, d, J=12.0 Hz), 5.04 (0.5H, d, J=12.0 Hz), 5.57-5.62 (1H, m), 5.64-5.90 (5H, m), 7.35-7.57 (9H, m), 7.70-7.87 (12H, m); MALDI-TOF MS, calcd. for C₉₆H₁₂₄O₁₇SiNa 1599.8506 (M+Na⁺), found 1599.8710.

### Example 3

Synthesis of acetal 37: To benzene solution (0.15 mL) of ABCDE ring diol 32 (7.7 mg, 13.4 μmol) and HIJKLM ring aldehyde 0.36 (17.6 μmol), Sc(OTf)₃ (1.9 mg, 3.9 μmol) was added at room temperature. After stirred at room temperature for 3 hours, reaction solution was diluted by ethyl acetate, NaHCO₃ aqueous solution was added and extracted by ethyl acetate for 3 times. Combined organic layer was washed by brine and dried on MgSO₄. After concentrated, purified by a flash column and compound 37 was obtained as diastereomer 1:1 mixture (10.1 mg, 6.4 μmol, 48%). ABCDE ring diol 32 (3.3 mg, 5.7 μmol) and HIJKLM ring aldehyde 36 (10.7 mg, 10.5 μmol) were recovered respectively in 43% and 60% yield.

Physical Property of Compound 37:

¹H NMR (500 MHz, CDCl₃) δ0.88-0.93 (3H, m), 1.01-1.08 (9H, m), 1.05 (21H, s), 1.10-1.15 (3H, m), 1.39 (0.5H, q, J=11.5 Hz), 1.40 (0.5H, q, J=11.5 Hz), 1.47-1.57 (5H, m), 1.68-2.02 (10H, m), 2.04-2.36 (8H, m), 2.52-2.59 (0.5H, m), 2.59-2.67 (0.5H, m), 2.64 (1H, ddd, J=16.0, 8.0, 4.0 Hz), 2.86 (1H, dd, J=8.5, 4.0 Hz), 2.96-3.02 (1H, m), 3.05-3.16 (4H, m), 3.19-3.31 (3H, m), 3.37 (0.5H, t, J=9.0 Hz), 3.37 (0.5H, t, J=9.0 Hz), 3.42 (0.5H, t, J=9.5 Hz), 3.43 (0.5H, t, J=9.5 Hz), 3.46-3.60 (4H, m), 3.67 (1H, d, J=10.0 Hz), 3.64-3.90 (7H, m), 4.00-4.14 (3H, m), 4.21 (0.5H, dd, J=11.0, 5.0 Hz), 4.30 (1H, dd, J=15.5, 5.5 Hz), 4.31-4.46 (0.5H, m), 4.67-4.70 (0.5H, m), 4.70 (0.5H, d, J=11.5 Hz), 4.71 (0.5H, d, J=11.5 Hz), 4.79 (1H, d, J=12.0 Hz), 4.81-4.87 (2H, m), 4.90 (0.5H, d, J=11.5 Hz), 4.98 (0.5H, d, J=12.0 Hz), 4.99 (0.5H, d, J=12.0 Hz), 5.03 (0.5H, d, J=12.0 Hz), 5.04 (0.5H, d, J=12.0 Hz), 5.57-5.62 (1H, m), 5.64-5.90 (5H, m), 7.35-7.57 (9H, m), 7.70-7.87 (12H, m); MALDI-TOF MS, calcd: C₉₆H₁₂₄O₁₇SiNa 1599.8506 (M+Na⁺), found:1599.8710.

### Example 4

Synthesis reactions of compounds 2 and 3 mentioned in above scheme 3; CH₂Cl₂ solution (0.5 mL) of compound 1 (16.8 mg, 10.7 μmol), TMSSPh (10 μL, 50 μmol), DTBMP (22 mg, 110 μmol) and activated MS4A (−10 mg) was cooled down to 0° C., and TMSOTf (17 μL, 90 μmol) was added. After stirred at room temperature for 2 hours, further stirred at 35° C. for 4 hours. Methanol (1 mL) and K₂CO₃ (80 mg) were added at room temperature and the mixture were stirred one night, then diluted by ethyl acetate. Organic layer was washed by brine, dried by MgSO₄, concentrated and purified by a flash column, thus O,S-acetal 2(6.9 mg, 4.1 μmol, 38%) was obtained and starting material 1 (3.9 mg, 2.5 μmol, 23%) was recovered.

Physical Property of Compound 2;

[α]_{D}²⁶−1.7±0.9 (c 0.355, CHCl₃); IR (film) ν3497, 3056, 2929, 2867, 1509, 1461, 1369, 1216, 1092, 1026, 883, 854, 815, 755 cm⁻¹; ¹H NMR (500 MHz, CDCl₃) δ0.90-0.93 (3H, m, Me57), 1.03 (21H, m, TIPS), 1.02-1.08 (9H, m, Me53, 54, 56), 1.12 (3H, d, J=7.5 Hz, Me55), 1.39 (1H, d, J=12.0 Hz, H40), 1.45-1.66 (5H, m, H10, 35, 37, 47, 48), 1.72 (1H, d, J=12.0 Hz, H32), 1.69-1.90 (6H, m, H22, 35, 36, 37, 50, 51), 1.92-2.01 (3H, m, H17, 50, 51), 2.04-2.13 (2H, m, H22, 32), 2.16-2.38 (6H, m, H4, 10, 17, 28, 40, 43), 2.47-2.54 (1H, m, H28), 2.64 (1H, ddd, J=16.0, 8.0, 4.0 Hz, H4), 2.86 (1H, dd, J=9.0, 4.5 Hz, H42), 2.96-3.12 (5H, m, H8, 9, 33, 38, 39), 3.17 (1H, ddd, J=11.0, 9.0, 4.5 Hz, H11), 3.24-3.30 (2H, m, H5, 34), 3.36 (1H, t, J=8.5 Hz, H6), 3.43 (1H, t, J=9.5 Hz, H46), 3.42-3.53 (4H, m, H7, 16, 21, 44), 3.67 (1H, d, J=9.5 Hz, H45), 3.73-3.80 (3H, m, H12, 23, 52), 3.80-3.90 (3H, m, H23, 41, 52), 3.93-3.98 (1H, m, H15), 3.95-4.07 (3H, m, H1, 15, 29), 4.20 (1H, dd, J=12.0, 4.5 Hz, H31), 4.31 (1H, dd, J=15.5, 5.5 Hz, H1), 4.38-4.42 (1H, m, H20), 4.75-4.81 (2H, m, NAP×2), 4.80 (1H, d, J=12.5 Hz, NAP), 4.83 (1H, d, J=12.5 Hz, NAP), 4.97 (1H, d, J=12.0 Hz, NAP), 5.02 (1H, d, J=12.0 Hz, NAP), 5.17 (1H, dd, J=11.0, 1.5 Hz, H27), 5.48-5.55 (1H, m, H18), 5.57-5.63 (2H, m, H14, 19), 5.70-5.80 (2H, m, H3, 14), 5.84-5.90 (1H, m, H2), 7.05-7.85 (26H, m, SPh, NAP×3); MALDI-TOF MS, calcd. for C₁₀₂H₁₃₀O₁₇SSiNa 1709.8696 (M+Na⁺), found 1709.8605.

PPTS (2.3 mg, 9.2 μmol) was added to CH₂Cl₂ solution (0.5 mL) of compound 1 (14.0 mg, 8.3 μmol) and ethylvinylether (EVE) (80 μL, 800 μmol) were added, then stirred at room temperature for 1.5 hours and the reaction was stopped by adding NaHCO₃ aqueous solution. The solution was extracted by ethyl acetate for 2 times, organic layer was washed by brine and dried by MgSO₄. After concentrated and purified by a flash column, EE ether (10.7 mg, 6.1 μmol) was obtained.

TBAF (61 μL, 1.0M THE solution, 61 μmol) was added to THF (0.2 mL) solution of EE ether (10.7 mg, 6.1 μmol) and stirred at 40° C. for 15 hours. Reaction solution was directly purified by a silica gel column and alcohol (10.6 mg) was obtained.

Methylpropiolate (11 μL, 120 μmol) and NMM (3 μL, 30 μmol) were added to CH₂Cl₂ solution (0.4 mL) of alcohol (10.6 mg) and stirred at room temperature for 9 hours. Reaction solution was directly purified by a silica gel column and acrylate (9.1 mg, 5.4 μmol, the total yield of above 2 processes is 89%), which is compound 3, was obtained.

Physical Property of Compound 3;

[α]_{D}²⁹ −0.2±1.1 (c 0.70, CHCl₃); IR (film)ν2928, 2873, 1715, 1642, 1623, 1509, 1455, 1372, 1331, 1291, 1087, 855, 817, 754 cm⁻¹; ¹H NMR (500 MHz, CDCl₃) δ0.87-0.92 (3H, m, Me57), 1.02-1.05 (3H, m, Me56), 1.06 (3H, d, J=7.0 Hz, Me54), 1.11 (3H, d, J=7.5 Hz, Me55), 1.22 (3H, t, J=7.0 Hz, EE), 1.26 (3H, s, Me53), 1.33 (3H, d, J=5.5 Hz, EE), 1.35-1.69 (7H, m, H10, 22, 35, 37, 40, 47, 48), 1.67 (1H, q, J=12.0 Hz, H32), 1.74-2.04 (8H, m, H17, 35, 36, 37, 50×2, 51×2), 2.14-2.43 (9H, m, H4, 10, 17, 22, 28×2, 32, 40, 43), 2.64 (1H, d, J=16.5, 8.0, 4.0 Hz, H4), 2.85 (1H, dd, J=9.5, 4.5 Hz, H42), 2.94-3.00 (1H, m, H38), 3.03-3.14 (4H, m, H8, 9, 33, 39), 3.15-3.21 (1H, m, H11), 3.24-3.31 (2H, m, H5, 34), 3.36 (1H, t, J=8.5 Hz, H6), 3.40 (1H, t, J=9.0 Hz, H46), 3.45-3.90 (14H, m, H7, 12, 16, 21, 23×2, 29, 41, 44, 45, 52×2, EE), 3.67 (3H, s, MeO), 3.96-4.08 (2H, m, H, 15), 4.26 (1H, dd) J=12.0, 5.0 Hz, H31), 4.30 (1H, dd, J=15.5, 5.5 Hz, H1), 4.41-4.46 (1H, m, H₂O), 4.68 (1H, d, J=12.5 Hz, NAP), 4.66-4.71 (1H, m, EE), 4.72 (1H, d, J=12.5 Hz, NAP), 4.80 (1H, d, J=12.0 Hz, NAP), 4.83 (1H, d, J=12.5 Hz, NAP), 4.98 (1H, d, J=12.0 Hz, NAP), 5.03 (1H, d, J=12.0 Hz, NAP), 5.11 (1H, dd, J=10.0, 2.5 Hz, H27), 5.24 (1H, d, J=13.0 Hz, H25), 5.45-5.81 (5H, m, H3, 13, 14, 18, 19), 5.83-5.89 (1H, m, H2), 7.14-7.86 (27H, m, H26, SPh, NAP×3); MALDI-TOF MS, calcd. for C₁₀₁H₁₂₂O₂₀SNa 1709.8148 (M+Na⁺), found 1709.8202.

Synthesis of compounds 4, 5 and 6 described in afore mentioned scheme 4; Toluene solution (1.8 mL) of compound 3 (9.1 mg, 5.4 μmol), Bu₃SnH(43 μL, 160 μmol) and AIBN (4.8 mg, 120 μmol) was degassed, then heated to 85° C. and stirred for 2.5 hours. Reaction solution was directly purified by a flash column and ester of compound 4 was obtained. Compound 4 was not further purified and used in the next reaction.

CH₂Cl₂ (0.5 mL) solution of compound 4 was cooled down to −80° C. and DIBAL (18 μL, 0.95 M hexane solution, 17 μmol) was added. Further, DIBAL (95 μL, 0.93 M hexane solution, 90 μmol) was introduced until starting material, compound 4, disappeared. After stirred at −80° C. for 2 hours, ethyl acetate was added and the reaction was stopped. The mixture was diluted by ethyl acetate and saturated NH₄Cl aqueous solution, added Rochelle salt and stirred at room temperature for 2 hours. The obtained solution was extracted by ethyl acetate for three times, and combined organic layer was washed by brine, dried by MgSO₄ and concentrated. Crude aldehyde was purified by a Florisil pad, and used for the next step reaction.

THF solution (0.2 mL) of triphenylphosphoniumbromide (24 mg, 67 mmol) was treated by HMDS (60 μL, 1M THF solution, 60 μmol) at 0° C., and the mixture was stirred at 0° C. for 15 minutes. THF solution (0.6 mL) of aldehyde was added to the solution and was stirred at 0° C. for 1 hour. Reaction was stopped by adding saturated NH₄Cl aqueous solution, and aqueous layer was extracted by ethyl acetate for 2 times. Combined organic layer was washed by brine and dried by MgSO₄. The obtained solution was concentrated, purified by a flash column and tetraene of compound 5 was obtained. Compound 5 was not further purified and used in the next reaction.

To the methanol (1 mL)-THF (0.5 mL) solution of EE ether compound 5, CSA (1.0 mg, 4 μmol) was added and stirred at room temperature for 3 hours. CSA (1.6 mg, 7 μmol) was further introduced and reaction solution was stirred at room temperature for 6 hours. Reaction was stopped by adding saturated NaHCO₃ aqueous solution to the mixture, and the aqueous layer was extracted by ethyl acetate for 2 times. Combined organic layer was washed by brine and dried by MgSO₄. After concentration and refining by a flash column, alcohol (3.3 mg, 2.2 μmol, total yield of said four processes was 41%), which is compound 6, was obtained.

Physical Property of Compound 6;

[α]_{D}²⁶ −7.1±2.3 (c 0.22, CHCl₃); IR (film) ν 3468, 2927, 2872, 1641, 1509, 1456, 1377, 1271, 1077, 854, 817, 755 cm⁻¹; ¹H NMR (500 MHz, CDCl₃) δ 0.87-0.92 (3H, m, Me57), 1.03-1.06 (3H, m, Me56), 1.10 (3H, d, J=7.5 Hz, Me54), 1.12 (3H, d, J=7.5 Hz, Me55), 1.26 (3H, s, Me53), 1.36-1.44 (1H, m, H40), 1.46-1.64 (6H, m, H10, 22, 35, 37, 47, 48), 1.62 (1H, q, J=12.0 Hz, H32), 1.73-2.05 (12H, m, H22, 25, 28×2, 32, 35, 36, 37, 50×2, 51×2), 2.10-2.39 (6H, m, H4, 10, 17, 25, 40, 43), 2.50-2.59 (1H, m, H17), 2.62-2.69 (1H, m, H4), 2.88 (1H, dd, J=9.0, 4.5 Hz, H42), 2.98-3.04 (1H, m, H38), 3.06-3.18 (6H, m, H8, 9, 21, 31, 33, 39), 3.18-3.24 (1H, m, H11), 3.29 (1H, td, J=9.5, 4.0 Hz, H5), 3.34-3.44 (4H, m, H6, 29, 34, 46), 3.47-3.56 (4H, m, H7, 16, 26, 44), 3.63-3.76 (4H, m, H₂O, 23×2, 27), 3.68 (1H, d, J=9.5 Hz, H45), 3.76 (1H, q, J=7.5 Hz, H52), 3.81-3.90 (4H, m, H12, 15, 41, 52), 4.03-4.09 (1H, m, H1), 4.32 (1H, dd, J=16.0, 6.0 Hz, H1), 4.78 (1H, d, J=12.5 Hz, NAP), 4.81 (1H, d, J=12.0 Hz, NAP), 4.84 (1H, d, J=12.0 Hz, NAP), 4.95 (1H, d, J=12.5 Hz, NAP), 4.97-5.03 (2H, m, H24′×2), 5.01 (1H, d, J=12.0 Hz, NAP), 5.06 (1H, d, J=12.0 Hz, NAP), 5.23 (1H, dd, J=11.0, 5.5 Hz, H19), 5.61-5.81 (5H, m, H3, 13, 14, 18, 24), 5.84-5.91 (1H, m, H2), 7.42-7.58 (9H, m, NAP×3), 7.77-7.87 (12H, m, NAP×3); MALDI-TOF MS, calcd. for C₉₁H₁₁₀O₁₇Na 1497.7641 (M+Na⁺), found 1497.7655.

Synthesis of compound 7 described in above mentioned scheme 4; CH₂CL₂ (0.2 mL)-DMSO (0.2 mL) solution of alcohol of compound 6 (3.3 mg, 2.2 μmol) and Et₃N (60 μL, 450 μmol) was cooled down to 0° C., and SO₃ pyridine complex (11 mg, 70 μmol) was added. Reaction solution was stirred at room temperature for 2 hours and diluted by ethyl acetate and saturated NH₄Cl aqueous solution. Aqueous layer was extracted by ethyl acetate for 2 times and combined organic layer was washed by brine, then dried by MgSO₄. After concentrated, crude aldehyde was purified by a Florisil pad and used for the next reaction.

THF solution (0.2 mL) of triphenylphosphoniumbromide (20 mg, 56 mmol) was treated by NaHMDS (55 μL, 1M THF solution, 55 μmol) at 0° C., and the mixture was stirred at 0° C. for 20 minutes. THF solution (0.4 mL) of ylide (56 μmol) prepared by same method was introduced to the reaction solution and stirred at 0° C. for 30 minutes. Reaction was stopped by adding saturated NH₄Cl aqueous solution and aqueous layer was extracted by ethyl acetate for 2 times. Combined organic layer was washed by brine and dried by MgSO₄. Said solution was concentrated and purified by a flash column pentaene, which is compound 7 (3.2 mg, 22 μmol) was obtained.

Physical Property of Compound 7;

[α]_{D}²⁶ −5.1±2.8 (c 0.21, CHCl₃); IR (film) ν 2927, 2872; 1641, 1509, 1456, 1377, 1272, 1090, 1026, 854, 817, 755 cm⁻¹; ¹H NMR (500 MHz, CDCl₃) δ 0.87-0.92 (3H, m, Me57), 1.04 (3H, d, J=6.0 Hz, Me56), 1.10 (3H, d, J=7.5 Hz, Me54), 1.12 (3H, d, J=7.5 Hz, Me55), 1.26 (3H, s, Me53), 1.36-1.44 (1H, m, H40), 1.46-1.62 (5H, m, H10, 35, 37, 47, 48), 1.63 (1H, q, J=11.5 Hz, H32), 1.72-2.05 (12H, m, H22, 25, 28×2, 32, 35, 36, 37, 50×2, 51×2), 2.10-2.39 (6H, m, H4, 10, 17, 25, 40, 43), 2.45-2.51 (1H, m, H22), 2.53-2.60 (1H, m, H17), 2.66 (1H, ddd, J=16.0, 8.0, 4.0 Hz, H4), 2.88 (1H, dd, J=9.0, 4.5 Hz, H42), 2.94 (1H, td, J=8.5, 2.5 Hz, H21), 2.98-3.03 (1H, m, H38), 3.07-3.15 (3H, m, H9, 33, 39), 3.14 (1H, t, J=9.0 Hz, H8), 3.17-3.23 (1H, m, H11), 3.22 (1H, dd, J=12.5, 4.5 Hz, H31), 3.29 (1H, td, J=9.5, 4.5 Hz, H5), 3.35-3.44 (4H, m, H6, 29, 34, 46), 3.49-3.58 (4H, m, H7, 16, 26, 44), 3.67 (1H, d, J=10.0 Hz, H45), 3.66-3.90 (7H, m, H12, 15, 20, 27, 41, 52×2), 4.02-4.09 (1H, m, H1), 4.32 (1H, dd, J=16.0, 6.0 Hz, H1), 4.78 (1H, d, J=12.5 Hz, NAP), 4.81 (1H, d, J=12.5 Hz, NAP), 4.84 (1H, d, J=12.5 Hz, NAP), 4.96 (1H, d, J=12.5 Hz, NAP), 4.97-5.03 (2H, m, H24′×2), 5.00 (1H, d, J=12.0 Hz, NAP), 5.06 (1H, d, J=12.0 Hz, NAP), 5.06-5.12 (2H, m, H23′×2), 5.25 (1H, dd, J=11.5, 5.5 Hz, H19), 5.60-5.90 (7H, m, H2, 3, 13, 14, 18, 23, 24), 7.42-7.58 (9H, m, NAP×3), 7.77-7.86 (12H, m, NAP×3); MALDI-TOF MS, calcd. for C₉₂H₁₁₀O₁₆Na 1493.7692 (M+Na⁺), found 1493.7682.

Synthesis of tris-NAP CTX3C 44 of Compound 8 Mentioned in Above Scheme 4;

In CH₂Cl₂ solution (2.2 mL) of pentaene (3.2 mg, 2.2 μmol), which is degassed compound 7, (PCy₃)₂Cl₂Ru═CHPh (Grubb's catalyst, 65 μL, 10 mM CH₂Cl₂ solution, 0.7 μmol) was added and stirred at 40° C. for 13 hours. Reaction was stopped by adding two drops of Et₃N, concentrated, purified by a flash column, and tris-NAP CTX3C (2.8 mg, 1.9 μmol, 90%), which is compound 8, was obtained.

Physical Property of Compound 8

IR (film) ν 2927, 2874, 1640, 1509, 1454, 1370, 1331, 1088, 1027, 855, 817, 756 cm⁻¹; ¹H NMR (500 MHz, CDCl₃) δ 0.90 (3H, d, J=6.0 Hz, Me57), 1.03 (3H, d, J=6.0 Hz, Me56), 1.06 (3H, d, J=7.5 Hz, Me54), 1.12 (3H, d, J=7.5 Hz, Me55), 1.23 (3H, s, Me53), 1.40 (1H, q, J=12.0 Hz, H40), 1.39-1.63 (5H, m, H10, 35, 37, 47, 48), 1.68 (1H, q, J=12.0 Hz, H32), 1.74-2.00 (7H, m, H35, 36, 37, 50×2, 51×2), 2.03-2.11 (2H, m, H28, 32), 2.16-2.38 (8H, m, H4, 10, 17, 22, 25, 28, 40, 43), 2.60-2.68 (2H, m, H4, 17), 2.70-2.90 (2H, m, H22, 25), 2.87 (1H, dd, J=9.0, 4.5 Hz, H42), 2.96-3.02 (1H, m, H38), 3.06-3.16 (5H, m, H8, 9, 31, 33, 39), 3.18-3.41 (4H, m, H5, 11, 27, 34), 3.37 (1H, t, J=8.5 Hz, H6), 3.39 (1H, t, J=9.5 Hz, H46), 3.47-3.60 (5H, m, H7, 16, 21, 29, 44), 3.67 (1H, d, J=9.5 Hz, H45), 3.64-3.90 (5H, m, H12, 20, 26, 41, 52), 3.75 (1H, q, J=8.0 Hz, H52), 3.95-4.00 (1H, m, H15), 4.01-4.07 (1H, m, H1), 4.30 (1H, dd, J=15.5, 6.0 Hz, H1), 4.74 (1H, d, J=12.5 Hz, NAP), 4.80 (1H, d, J=12.0 Hz, NAP), 4.81 (1H, d, J=12.5 Hz, NAP), 4.84 (1H, d, J=12.0 Hz, NAP), 4.98 (1H, d, J=12.0 Hz, NAP), 5.03 (1H, d, J=12.0 Hz, NAP), 5.57-5.62 (1H, m, H14), 5.67-5.90 (7H, m, H2, 3, 13, 18, 19, 23, 24), 7.42-7.56 (9H, m, NAP×3), 7.75-7.85 (12H, m, NAP×3); MALDI-TOF MS, calcd. for C₉₀H₁₀₆O₁₆Na 1465.7379 (M+Na⁺), found 1465.7493.

Synthesis of CTX3C, Which is Aimed Product;

DDQ (1.1 mg, 2.2 μmol) was added to (CH₂Cl₂)₂ (0.8 mL)-H₂O (40 μL) solution of tris-NAP CTX3C (1.4 mg, 1.9 μmol), which is compound 8, and stirred at room temperature for 3 hours. Reaction was stopped by adding saturated Na₂S₂O₃ aqueous solution, then diluted by ethyl acetate and saturated NaHCO₃ aqueous solution. Aqueous layer was washed by ethyl acetate for 3 times and combined organic layer was washed by brine, then dried by MgSO₄. Solution was concentrated and passed through Florisil column, then crude CTX3C was purified by HPLC and synthesis CTX3C (0.53 mg, 0.52 μmol, 54%) was obtained.

Physical Property of CTX3C;

¹H NMR (600 MHz, C₅D₅N, 25° C.): δ 0.91 (3H, d, J=7 Hz, Me54), 0.97 (3H, d, J=7 Hz, Me57), 1.23-1.32 (9H, m, Me53, 55, 56), 1.49-1.56 (1H, m, H35), 1.60 (1H, dq, J=10, 7 Hz, H48), 1.63-1.98 (11H, m, H10, 32, 35, 36, 37, 40, 47, 50×2, 51×2), 2.02 (1H, brd, J=15 Hz, H37), 2.23-2.33 (4H, m, H17, 22, 25, 32), 2.40-2.61 (6H, m, H4, 10, 28×2, 40, 43), 2.67 (1H, ddd, J=16, 8, 4 Hz, H4), 2.82-2.88 (1H, m, H17), 2.95-3.08 (2H, m, H22, 25), 3.20 (1H, dd, J=10, 4 Hz, H42), 3.17-3.23 (1H, m, H38), 3.28-3.39 (4H, m, H9, 31, 33, 39), 3.43 (1H, t, J=9 Hz, H8), 3.43 (1H, t, J=9 Hz, H11), 3.40-3.50 (2H, m, H5, 34), 3.53 (1H, t, J=9 Hz, H6), 3.55-3.75 (4H, m, H16, 21, 26, 27), 3.85-3.90 (2H, m, H52×2), 3.94 (1H, t, J=10 Hz, H46), 4.04 (1H, brd, J=10 Hz, H45), 4.05-4.20 (6H, m, H1, 7, 12, 15, 20, 29), 4.21 (1H, brs, H44), 4.33 (1H, dd, J=16, 6 Hz, H1), 4.45-4.50 (1H, m, H41), 5.71-5.77 (1H, m, H3), 5.81-5.89 (3H, m, H2, 13, 18), 5.93 (1H, brd, J=13 Hz, H14), 5.98 (1H, dd, J=11, 5 Hz, H19), 6.00-6.06 (2H, m, H23, 24); MALDI-TOF MS calcd for C₅₇H₈₂O₁₆Na [M+Na]⁺ 1045.5501; found 1045.5553; CD (MeOH, 1.0×10⁻⁵ M) λₑₓₜ 203 nm (Δε−5), [natural 2 (MeOH, 1.0×10⁻³ M) λₑₓₜ 203 nm (Δε−7)].

### POSSIBILITY FOR THE INDUSTRIAL USE

As mentioned above, by the new route for synthesis of ciguatoxin of the present invention, since the deprotection of three protecting groups by mild and substrate specific oxidation condition is possible at the final process, more effective synthesis of CTX3C becomes possible, compared with the case when conventional Bn protecting group is used. Therefore, the activation of various physiological studies and the accompanied development of new technique and excellent effects are predicted.

Illustration of abbreviations in the present specification;

| | |
|---|---|
| NAP | 2-naphthylmethyl |
| DTBMP | 2,6-di-butyl-4-methylpyridine |
| TMSOTf | trimethylsilyl trifluoromethanesulfonate |
| TMSSPh | phenylthiotrimethylsilane |
| Me | methyl |
| TIPS | triisopropylsilyl |
| Ph | phenyl |
| EE | ethoxyethyl |
| EVE | ethylvinylether |
| PPTS | pyridinium p-toluenesulfonate |
| TBAF | tetrabutylammonium fluoride |
| NMM | 4-methylmorphorine |
| PMBM | 4-methoxybenzyloxymethyl |
| AIBN | α,α′-azobis(isobutyronitrile) |
| DIBAL | diisobutylaluminum hydride |
| NaHMDS | sodium bis(trimethylsilyl)amide |
| CSA | 10-camphorsulfonic acid |
| THF | tetrahydrofuran |
| DMSO | dimethylsulfoxide |
| Cy | cyclohexyl |
| DDQ | 2,3-dichloro-5,6-dicyano-1,4-benzoquinone |
| Grubbs | benzylidene-bis(tricyclohexylphosphine)dichlororuthenium |
| catalyst | |
| Tf | trifluoromethanesulfonyl |

1. An intermediate for synthesis of ciguatoxins comprising, a compound represented by chemical formula 1 which uses naphthyl group as a protecting group for hydroxyl groups locating at C7, C29 and C44 of said compound represented by chemical formula 1, 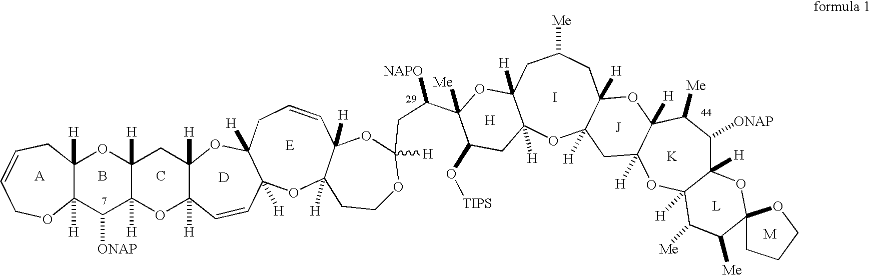
wherein, abbreviations are mentioned in the specification.

2. A method for synthesis of the compound indicated by the chemical formula 1 comprising, coupling reaction of two rings wherein one is a compound composing ABCDE ring segment of chemical formula 2 and another one is a compound composing HIJKLM ring segment of chemical formula 3, 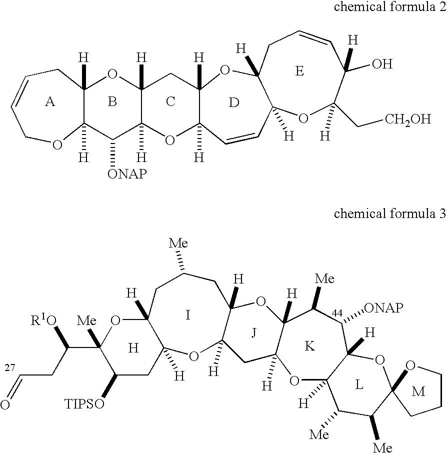
wherein R¹ in chemical formula 2 is NAP.

3. An intermediate represented by chemical formula 2 which synthesizes the compound represented by chemical formula 1.

4. An intermediate represented by chemical formula 3 which synthesizes the compound represented by chemical formula 1.

5. A method for synthesis of the aimed compound CTX3C by following scheme 4, 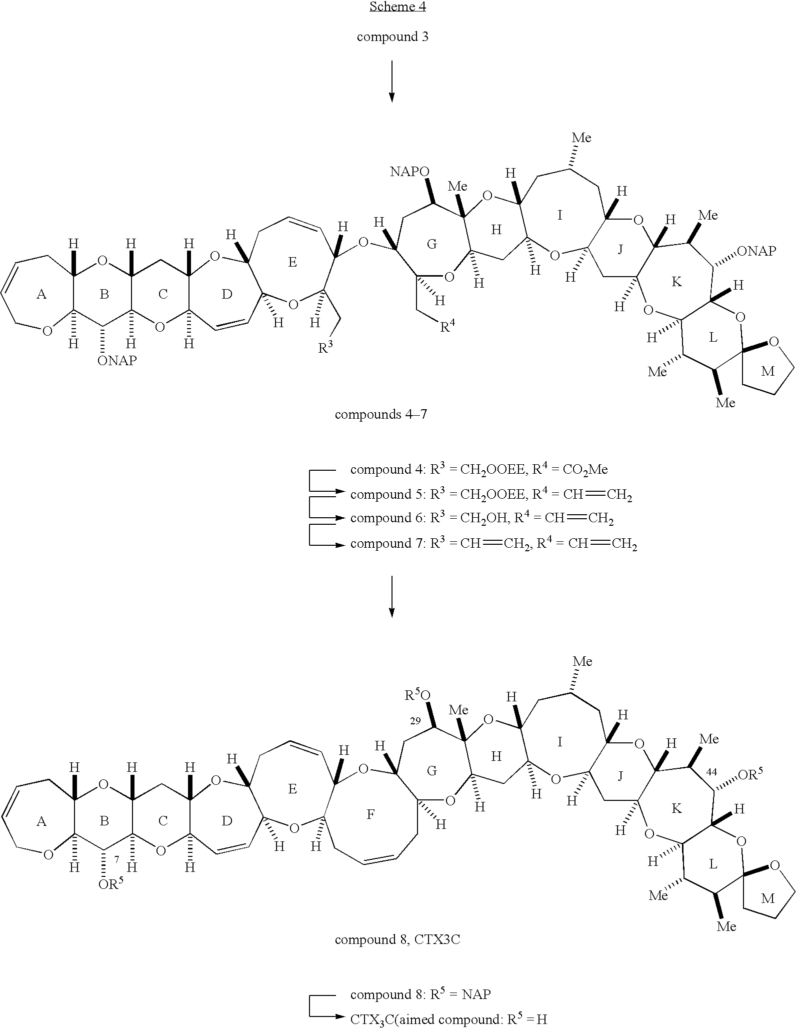
using acetal compound, whose protecting groups for hydroxyl groups locating at C7, C29 and C44 are naphthylmethyl (NAP) group, represented by compound 1 which corresponds to the compound represented by chemical formula 1 of claim 1 comprising,
process 1; which obtains following O,S-acetal compound 2, by placing said compound 1 under the presence of 2,6-di-t-butyl-4-methylpridine (DTBMP), treating by trimethylsilyl trifluoromethanesulfonate (TMSOTF) and phenylthiotrimethylsilane (TMSSPh) with maintaining protecting group of NAP group,
process 2; reaction represented by following scheme 3 consisting of the process to obtain following compound 3, protecting primary hydroxyl group locating at C23 of the compound 2 with ethoxyethyl group, introducing α,β-unsaturated ester to secondary hydroxyl group locating at C31, and 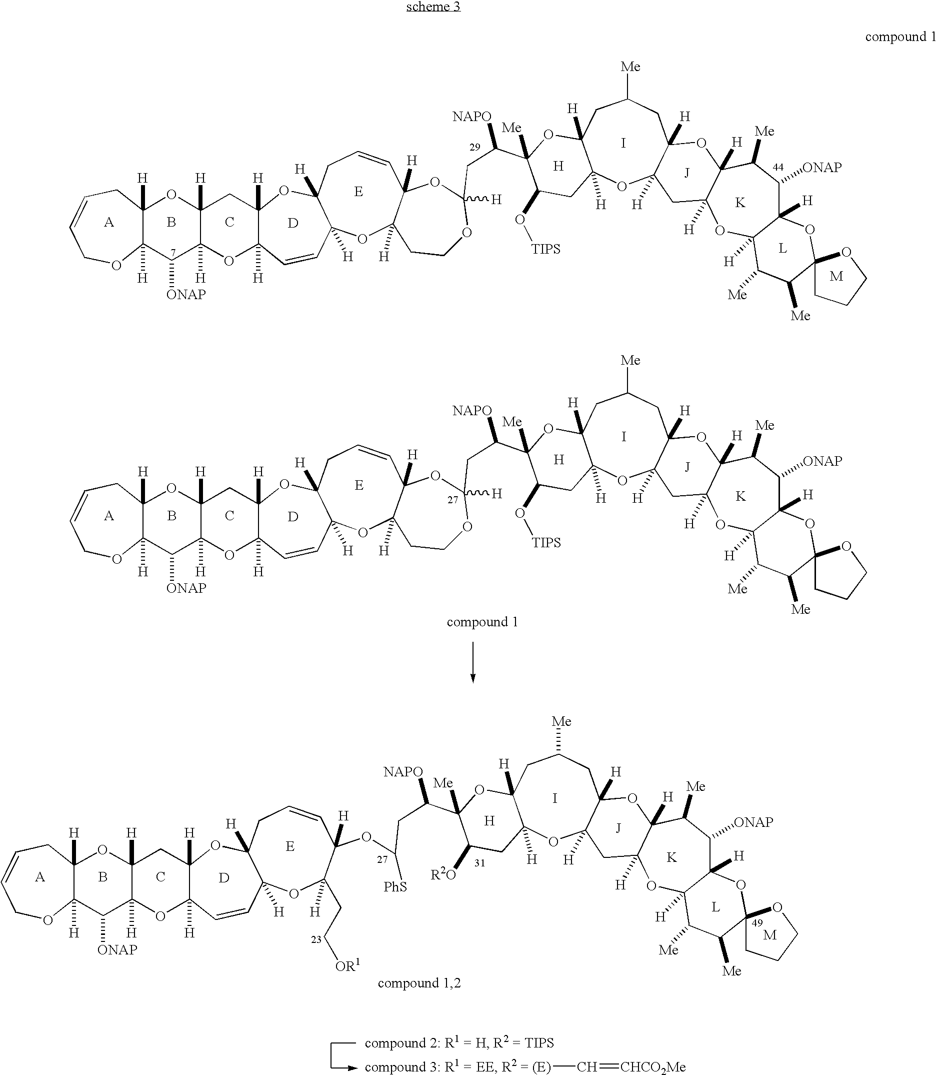
process 3; process to synthesize the compound 4 mentioned in the scheme 4 forming G ring segment by stereoselective radical cyclization,
process 4; process to synthesize olefin compound 5 mentioned in the scheme 4 by reducing ester part of the compound 4 and by Wittig reaction,
process 5; process to obtain the compound 6 mentioned in the scheme 4 by removing ethoxyethyl group by treating the compound 5 under acid condition,
process 6; process to synthesize following compound 7 mentioned in the scheme 4, which is ring closing olefin methathesis substrate, by oxidizing the compound 6 and providing it to Wittig reaction.
process 7; process to synthesize the compound 8 mentioned in the scheme 4, which is tris NAP-CTX3C, by forming F ring from compound 7 using Grubbs catalyst indicated by (PCY₃)₂Cl₂Ru═ChPh [benzylidene bis(tricyclohexylphosphine)dichlororuthenium], and final process 8; process to make the NAP deprotecting group reaction of the compound 8 by DDQ under oxidation condition.

6. A compound represented by chemical formula A, 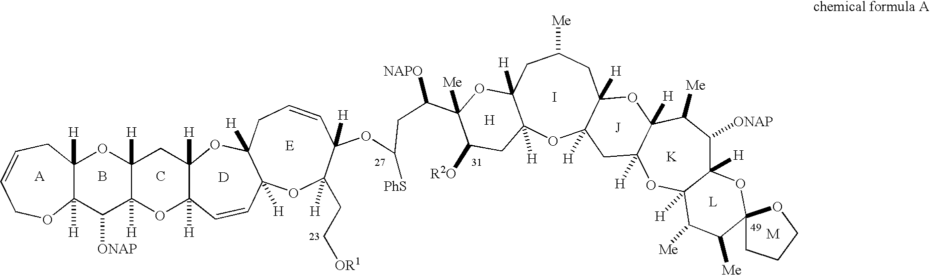
wherein, R¹ is H or EE, R² is TIPS or CH═CHCOO₂Me.

7. A compound represented by chemical formula B, 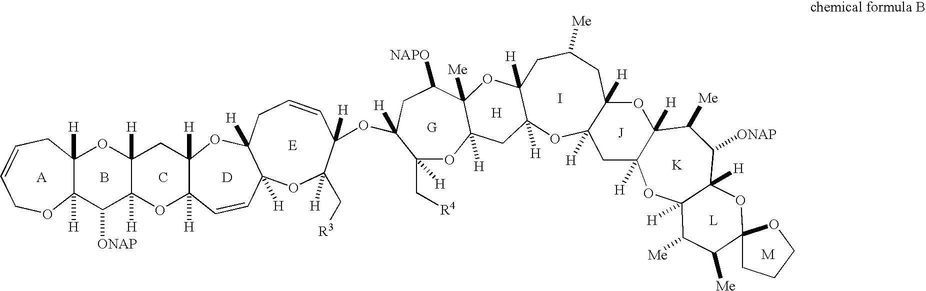
wherein, R³ is CH₂OEE, CH₂OH or CH═CH₂, R⁴ is COOMe or CH═CH₂.

8. A compound represented by chemical formula C, 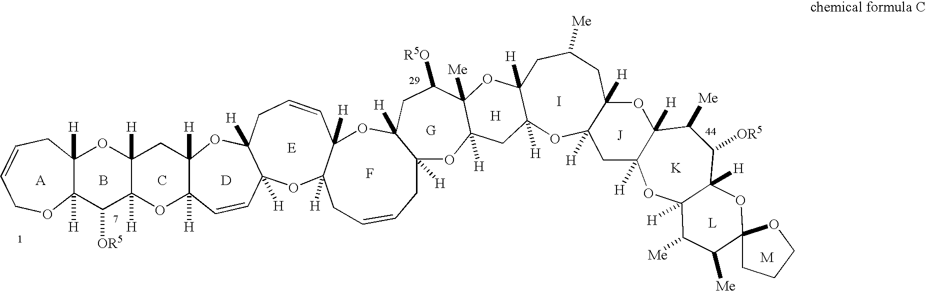
wherein, R⁵ is NAP.
